(19) United States
(12) Patent Application Publication
(10) Pub. No.: US 2011/0280907 A1
(43) Pub. Date: Nov. 17, 2011

(54) METHOD AND SYSTEM FOR BUILDING A PHYLOGENY FROM GENETIC SEQUENCES AND USING THE SAME FOR RECOMMENDATION OF VACCINE STRAIN CANDIDATES FOR THE INFLUENZA VIRUS

(75) Inventors: Alice Carolyn Mchardy, Düsseldorf (DE); Lars Steinbruck, Düsseldorf (DE)

(73) Assignee: MAX-PLANCK-GESELLSCHAFT ZUR FORDERUNG DER WISSENSCHAFTEN E.V., MUNICH (DE)

(21) Appl. No.: 13/130,983

(22) PCT Filed: Nov. 25, 2009
(86) PCT No.: PCT/EP2009/008397

§ 371 Date: Aug. 1, 2011

(30) Foreign Application Priority Data
Nov. 25, 2008 (EP) 08020435.7

(51) Int. Cl. A61K 39/145; G06F 19/14; A61P 31/16

(52) U.S. Cl. 424/209.1; 702/19

(57) ABSTRACT

A computer-implemented method and a computer system for identifying a phylogenetic tree from a plurality of biological sequences is provided. Each biological sequence is associated with a sampling date. First, the plurality of biological sequences is aligned and a distance matrix is obtained. Then, a subset of these sequences without any duplicated sequences is selected and a directed graph representation of the subset of biological sequences is generated based the associated sampling dates. Then, a minimum spanning tree is computed from the weighted directed graph representation. Then, in an iterative procedure, the sequences of unsampled evolutionary intermediates are inferred from mutation patterns that reflect the difference in sequence between the nodes in the minimum spanning tree. The new sequences are added with associated time stamps to the sequence set. Then, sets of identical sequences are removed. Then, an optimum branching is recomputed. This step is repeated until no new intermediates are found. In the final step, the sequences that have been set aside in the initializing step are added to the plurality of sequences derived in the update step. From this plurality of sequences an optimum branching is computed and identified as the phylogenetic tree. Amino acid changes repeatedly occurring on the internal branches of the obtained tree can be used to identify sequences and associated viral isolates suitable as vaccine strains for the following influenza season.

### BACKGROUND OF THE INVENTION

1. Field of the Invention

The invention relates to the construction of phylogenetic trees and more particular to their application to identify vaccine strain isolates for the influenza virus.

2. Description of the Related Art

Methods for constructing phylogenetic trees are of central importance in the study of viral diseases. Phylogenies indicate the importance of individual sequence sites and mutation events in evolution, allow identification of fast evolving sites and sites under positive or purifying selection, and give insight into the timing and direction of mutation events. Phylogenies and the mutation events mapping onto their internal edges are studied in the process of vaccine strain selection for seasonal influenza, to monitor viral geographic spread, the appearance and prevalence of drug-resistant viral variants, the detection of host transfer events for viruses from different host reservoirs and to identify the emergence of human transmissible influenza strains with the potential to cause a pandemic.

Conventionally, phylogenetic techniques search for tree topologies under a given optimality criterion with tree rearrangement operations such as nearest-neighbor interchanges, subtree pruning and regrafting or tree bisection and reconnection. This is allows the construction of phylogenies from sets of sequences at arbitrary evolutionary distances, but becomes computationally very demanding for large sequence sets.

For instance, S. Khumar et al. “MEGA3: Integrated software for Molecular Evolutionary Genetics Analysis and sequence alignment”, BRIEFINGS IN BIOINFORMATICS, VOL 5, NO 2, pages 150-163, describes the Molecular Evolutionary Genetics Analysis (MEGA) software, with its focus on facilitating the exploration and analysis of the DNA and protein sequence variation from an evolutionary perspective. The third major release, MEGA3, contains facilities for automatic and manual sequence alignment, web-based mining of databases, inference of the phylogenetic trees, estimation of evolutionary distances and testing evolutionary hypotheses. Phylogenetic trees can be constructed with distance-matrix methods such as neighbor-joining or UPGMA (Unweighted Pair Group Method with Arithmetic mean), based on genetic distances inferred from multiple sequence alignments. UPGMA is known to not perform well when evolution is non-clocklike, i.e. when the rate of evolution varies along different branches. Neighbor-joining phylogenies are often used as a starting point for methods such as described below, to further improve the tree under a given optimality criterion.

More advanced methods use optimality criteria such as maximum parsimony (least number of overall mutations), maximum likelihood or Bayesian inference under an explicit model of sequence evolution. Available software for inference of phylogenies under these criteria include PAUP (Swofford, D. L. “PAUP: Phylogenetic Analysis Using Parsimony (*and Other Methods”), Version 4.0d64 ed, Sinauer Associates, Inc. Publishers, Sunderland, Mass.), GARLI (Zwickl, D. J. “Genetic algorithm approaches for the phylogenetic analysis of large biological sequence datasets under the maximum likelihood criterion”. Ph.D. dissertation, The University of Texas at Austin., PhyML (Guindon and Olivier Gascuel “A simple, fast and accurate method to estimate large phylogenies by maximum-likelihood”, SYSTEM BIOLOGY VOL 52, pages 696-704) and BEAST (Drummond A J and Rambaut A “BEAST: Bayesian evolutionary analysis by sampling trees” BMC EVOLUTIONARY BIOLOGY VOL 7, page 214).

A characteristic hallmark of sequence data for the influenza virus is the sampling density, with hundreds of isolates from the major circulating subtypes being sequenced every year. This is reflected in the low genetic and accordingly, evolutionary divergence of the corresponding sequence isolates.

The early identification of the next predominant influenza strain is important with regard to the problem of selecting a viral strain for production of the influenza vaccine. This strain should be well matched antigenically to the predominant viral variant in the following influenza season. Due to the time requirements of the production and distribution process, this strain has to be identified almost one year in advance from available genetic, antigenic and phenotypic surveillance data for seasonal influenza A viruses from around the globe. The present invention provides methods for predicting forthcoming predominant influenza strains from available data sets.

### SUMMARY OF THE INVENTION

It is the object of the present invention to provide phylogenies and genetic markers of viral fitness for genetic sequence data and their use for the selection of vaccine strains for seasonal influenza.

This object is solved by the subject matter of the independent claims.

Preferred embodiments are defined by the dependent claims.

In an embodiment, a method for identifying a phylogenetic tree for a plurality of biological sequences is provided. Each biological sequence is associated with a time stamp. First, distances between biological sequences of the plurality of biological sequences are obtained. Then, a weighted directed graph representation of the plurality of biological sequences is generated based on the obtained distances and the sampling dates associated with the plurality of biological sequences. Finally, a minimum spanning tree, MST, of the generated graph is computed and identified as the phylogenetic tree.

In another embodiment, a system for identifying a phylogenetic tree for a plurality of biological sequences, wherein each biological sequence is associated with a time stamp, includes a data preparer and a phylogeny identifier. The data preparer is communicatively coupled to the phylogeny identifier. The data preparer is adapted to obtain and output distances between biological sequences of the plurality of biological sequences. The phylogeny identifier comprises a graph generator and an minimum spanning tree, MST, identifier. The graph generator is adapted to generate a graph representation of the plurality of biological sequences based on the output distances from the data preparer and the time stamps associated with the plurality of biological sequences. The minimum spanning tree identifier is further adapted to compute an minimum spanning tree of the generated graph and identify the computed minimum spanning tree as the phylogenetic tree.

In a further embodiment, the computed phylogenetic tree forms the basis for the preparation of a vaccine, in particular to identify the expected viral strain for a forthcoming virus outbreak, which in turn forms the basis for the vaccine preparation.

In another embodiment, the preparation of a vaccine against a fast-evolving entity is based on a phylogenetic tree and includes the selection of an individual sequence and associated isolate from which the sequence was obtained for the entity; and the preparation of a vaccine based on the selected individual isolate.

Another embodiment pertains to the vaccine produced by the foregoing method.

In a further embodiment, the selection of an individual isolate of the fast-evolving entity comprises the steps of (i) computation of a phylogenetic tree from sequences available up to the point in time where the decision is to be made, (ii) identifying the number of repeated amino acid mutations occurring in each isolate and (iii) the selection of an isolate as a vaccine candidate when (a) the isolate sequence bears the maximum number of repeated amino acid mutations that occurred within a time-span of 4 years before the season in which the vaccine is to be produced, or (b) the isolate sequence bears the maximum number of repeated amino acid mutations that are located in antigenic sites and occurred within a time-span of 4 years before the season in which the vaccine is to be produced, or (c) the isolate sequence bears the maximum number of repeated amino acid mutations that cluster in the phylogeny, are located in antigenic sites and occurred within a time-span of 3 years before the season in which the vaccine is to be produced, or (d) the isolate sequence bears the maximum number of repeated amino acid mutations that cluster in the phylogeny, are located in sites determined experimentally to change the viral phenotype and occurred within a time-span of 4 years before the season in which the vaccine is to be produced.

### BRIEF DESCRIPTION OF THE DRAWINGS

The accompanying drawings are incorporated into and form a part of the specification for the purpose of explaining the principles of the invention. The drawings are not to be construed as limiting the invention to only the illustrated and described examples of how the invention can be made and used. Further features and advantages will become apparent from the following and more particular description of the invention, as illustrated in the accompanying drawings, wherein:

FIG. 1 illustrates an exemplary method for constructing a phylogenetic tree;

FIGS. 2a to 2c illustrates an exemplary method for generating a graph representation for a set of biological sequences;

FIG. 3 illustrates an exemplary system adapted to construct a phylogenetic tree;

FIGS. 4a to 4c shows an exemplary phylogenetic tree constructed in accordance with an embodiment of the invention;

FIG. 5 shows the timeline and relative frequency for antigenic clades between 1993 and 2007 inferred from the phylogeny based on signature mutations of antigenic types;

FIG. 6 shows two types of mutation patterns which indicate unsampled evolutionary intermediate sequences.

FIG. 7 shows an exemplary phylogenetic tree with original nodes in white and inferred intermediates hatched (center, ATGAC); sequence substitutions with respect to the root of the tree (ACGAC) are underlined.

FIG. 8 shows an exemplary method for inferring new intermediates and repeated substation patterns, wherein node V₀ is a parent of an internal node A and nodes V₁, V₂, . . . , V_{N} are children of A. Each Xᵢ is a set of substitutions whereas a substitution is defined as a triple (char from, position, char to) that denotes a position which differs between sequences A and Vᵢ as seen in direction from A to neighboring node Vᵢ (e.g. C2T would signify that C at position 2 of a sequence in a parent node is substituted with T in the child node). The negative sign before X₀ denotes the fact that −X₀ is a set of point mutations as seen from V₀ to A whereas X₀ is a set of point mutations as seen from A to Vₒ.

FIGS. 9 and 10 show two examples for the method of inferring intermediates and repeated substitution patterns, wherein the left hand side of the Figure represents the initial situation (input tree) and the right hand side of the Figure represents the calculated phylogeny with inferred intermediates (output tree). The repeated substitutions (C2T) are encircled in dashed lines, original nodes are given in white and inferred intermediate nodes are hatched (inferred intermediated are ATGAC in FIG. 9, and ATGTC in FIG. 10); sequence substitutions that mark repeated substitutions in the input tree are underlined.

FIGS. 11A and B show a gene allele predominance plot where alleles that reach a prevalence of more than 90% in frequency for a given year are in solid lines and marked with arrows, whereas all others are shown in dashed lines and gray. FIG. 11A shows the data for the Northern hemisphere isolates, FIG. 11B shows the data for the Southern hemisphere isolates. Predominant alleles are named according to literature names of predominant influenza A (H3N2) variants between 1998 and 2009.

FIG. 12 shows a topological accuracy evaluation for different algorithms, wherein pTree is the pattern-based stochastic search for maximum parsimony of the present invention, PHYML is the maximum likelihood method of Guindon and Gascuel (Systematic Biology 2 (2003):696-704), in combination with subsequent branch length optimization with Garli (D. Zwickl, PhD Thesis, The University of Texas at Austin 2006), PAUP NJ is the Neighbor Joining algorithm, PAUP ML is the maximum likelihood method and PAUP MP is the maximum parsimony method of PAUP (Swofford, D., 2002, Phylogenetic Analysis Using Parsimony (*and other methods). Version 4. Sinauer Associates; Sunderland, Mass.).

### DETAILED DESCRIPTION OF THE INVENTION

The illustrative embodiments of the present invention will be described with reference to the figure drawings, wherein like elements and structures are indicated by like reference numbers.

The present invention provides a new method and system for building phylogenetic trees (phylogenies) from biological sequences. For densely sampled sequence sets from a single evolving population such as the H3N2 subtype of seasonal influenza, a phylogeny which is identical or a close approximation in cost to a conventional maximum parsimony solution may be identified in at most quadratic time, which makes it applicable to data sets two to three orders of magnitude larger than what can be processed with conventional maximum parsimony techniques. The inferred trees may furthermore be multifurcating instead of bifurcating which is a more realistic representation of evolutionary relationships. Embodiments of the present invention are first to guarantee finding such a phylogeny, which is advantageous in particular for the study of fast evolving entities such as viruses from large sets of sequences.

A characteristic hallmark of sequence data for the influenza virus is the sampling density, with hundreds of isolates from the major circulating subtypes being sequenced every year. This is reflected in the low genetic and accordingly, evolutionary divergence of the corresponding sequence isolates.

The phylogenetic method of the present invention is suited for densely sampled sequences with low genetic divergence. One of the key differences between the present invention and common techniques is that instead of searching the tree space by tree-rearrangement operations for an optimal solution, ancestral intermediates are iteratively inferred based on characteristic mutation patterns in the phylogeny. For sequence sets of low genetic divergence such as those sampled from a single species, viral subtype or replicating cell line, this procedure allows to identify a phylogeny which is identical or a close approximation in cost to a maximum parsimony solution. The computing time rises at most quadratically with the number of input sequences, which makes the invention applicable to sets of sequences several orders of magnitude larger than what can be processed with conventional parsimony techniques. A further distinction is that the present invention allows to infer multifurcating tree topologies, which are a more realistic representation of the evolutionary relationships than bifurcating topologies returned by conventional approaches.

By applying methods and systems of the present invention to sequence data of seasonal influenza, subtype H3N2, repeatedly occurring amino acid changes in the inferred phylogeny are found that are significantly associated with the process of antigenic drift. These changes allow recommendation of suitable vaccine strains in seasons where an update of the vaccine components is required.

In short, an embodiment of the present invention provides a computer-implemented method and a computer system for identifying a phylogenetic tree from a plurality of biological sequences. Each biological sequence is associated with a sampling date. First, the plurality of biological sequences is aligned and a distance matrix is obtained. A subset of these sequences without any duplicated sequences is selected and a directed graph representation of the subset of biological sequences is generated based on the associated sampling dates. In some embodiments, the directed graph representation may be generated for the entire plurality of biological sequences. Then, a minimum spanning tree is computed from the weighted directed graph representation. In some embodiments, the sequences of unsampled evolutionary intermediates may be inferred in an update step in an iterative procedure from mutation patterns that reflect the difference in sequence between the nodes in the minimum spanning tree. The new sequences are added with associated time stamps to the sequence set. Then, sets of identical sequences may again be removed and an optimum branching (minimum spanning tree) may be recomputed. This step may be repeated until no new intermediates are found. In a final step, the sequences that have been set aside, if any, may be added to the plurality of sequences derived in the update step. From this plurality of sequences an optimum branching is computed and identified as the phylogenetic tree. Amino acid changes repeatedly occurring on the internal branches of the obtained tree can be used to identify sequences and associated viral isolates suitable as vaccine strains for the following influenza season.

An “isolate” is a sample of an entity that was isolated from a single source. The isolate represents a clonal subset of the entity, i.e. the isolate's sequence is derived from genetically identical individuals of the entity and the sequence of the isolate is unambiguous.

An “entity” is any organism, virus or replicating cell line. The entity can be a pathogen and either a microorganism or a virus. Microorganisms include bacteria, fungi, archaea, and protists, as well as microscopic plants such as plankton and animals such as amoeba. In an embodiment, the entity is a replicating cell line, such as a cancer cell line. In an embodiment, the entity is a virus. The virus may belong to the family of any one of Adenoviridae, Picornaviridae, Herpesviridae, Hepadnaviridae, Flaviviridae, Retroviridae, Orthomyxoviridae, Paramyxoviridae, Papovaviridae, Rhabdoviridae, or Togaviridae. Further, the entity may be selected from Influenzavirus A, Influenzavirus B, Influenzavirus C, Thogotovirus and Isavirus (all Orthomyxoviridae). In preferred embodiments the entity may subtypes of influenza virus A, such as H1N1, H1N2, H2N2, H3N1, H3N2, H3N8, H5N1, H5N2, H5N3, H5N8, H5N9, H7N1, H7N2, H7N3, H7N4, H7N7, H9N2 or H10N7. A “fast-evolving entity” is an entity that is capable of reproduce itself or, in the case of a virus, being reproduced within a short period of time. A short period of time is, for example, less than one week or less than one day.

The phylogenetic tree of the present invention can be multifurcating with nodes that have an arbitrary number of direct descendants. Thus, phylogenies for data with multiple or just one direct descendant(s) to any ancestral sequence can be identified.

The present invention also allows an intuitive visualization of evolutionary relationships along with epidemiological and experimental information and their study on the phylogenetic tree. The method and system of the present invention were evaluated with a study of the major surface protein of the influenza A virus (subtype H3N2) over the time span from 1993 to 2007. H3N2 is the most rapidly evolving variant of endemic influenza and was introduced in the human population in 1968 with the Hong Kong pandemic. Phylogenetically, influenza A belongs to the family of Orthomyxoviridae and features a single-stranded RNA genome composed of eight distinct segments totaling approximately 11 kb in length. Through continuous change (antigenic drift) in the epitope sites of the major surface proteins, hemagglutinin (HA) and neuraminidase (NA), the virus is able to evade immune recognition in previously primed individuals. Thus, one may incur multiple infections over the course of several years, despite existing immunity to older variants, and repeated updates of vaccines with novel strains are required. Due to the complex interplay of factors shaping antigenic drift and viral fitness (high mutation rates, global seasonal spread and interaction with the inherently variable acquired immune response), understanding the process of antigenic drift is still a key target in influenza research today.

A phylogenetic tree or phylogeny, sometimes also called an evolutionary tree, is a tree showing the evolutionary relationships among various biological isolates that are believed to have a common ancestor. A rooted phylogenetic tree is a directed tree with a unique node corresponding to the most recent common ancestor of all the biological isolate sequences at the leaves or internal nodes of the tree. Unrooted trees, on the other hand, illustrate the relatedness of the nodes without making assumptions about common ancestry.

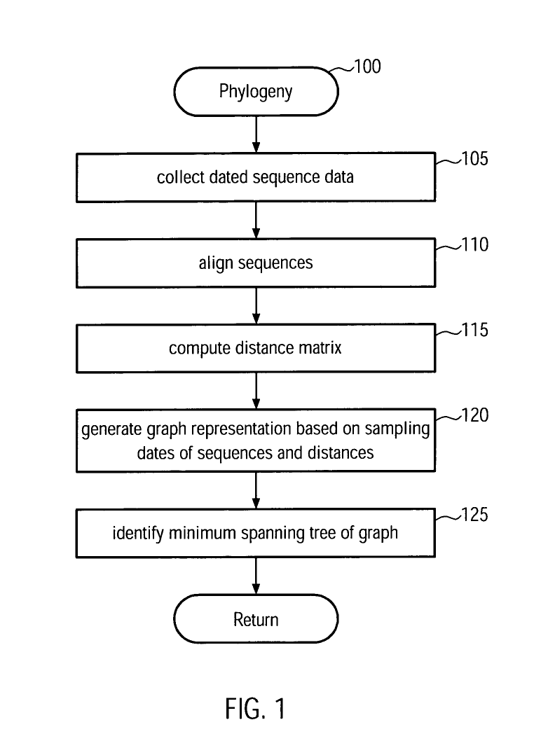

Referring to FIG. 1, unlike conventional techniques for identifying phylogenetic trees, embodiments of the present invention may provide a multifurcating instead of a bifurcating phylogenetic tree and is applicable to sequence sets two to three orders of magnitude larger than what can be processed with conventional maximum parsimony techniques.

Embodiments of the present invention may be applied to identify a phylogenetic tree for any set of biological sequences of interest. A biological sequence (also referred to as sequence) is the primary structure of a biopolymer. For instance, a biological sequence may be the order of the nucleotide bases in a DNA or RNA molecule or the order of amino acids in a protein. The sequences that are subject to the phylogenetic analysis may be ribonucleic acid sequences (RNA sequences), deoxyribonucleic acid sequences (DNA sequences) or amino acid sequences (protein sequences).

Accordingly, at step 105, data regarding biological sequences of the biological entity of interest, such as a virus or any other entity, is collected. In an embodiment, dated sequence data is collected, i.e. each biological sequence is associated with a sampling date or a time stamp. The sequences can be derived from public databases or from any other source. This may be done by accessing a central database such as the Influenza Sequence Database at NCBI (National Center for Biotechnology Information) or a database of the WHO (World Health Organization), where biological sequence data is stored.

In an embodiment, the biological sequences are nucleic acid sequences encoding a protein, or protein sequences of the entity. The protein may be at least partially exposed to the surface of the fast-evolving entity and may contain at least one antigenic site. An antigenic site is an epitope of a protein, said epitope being able to elicit an immune response of the host to said epitope. The epitope may be surface-exposed. In a preferred embodiment, the virus may be an Influenza virus and the sequences used for the determination of the phylogeny may be selected from hemagglutinin (HA) or neuraminidase (NA).

In an embodiment, the sampling date/time stamp associated with a biological sequence may correspond to the date on which the biological sequence has been determined from the isolate of the biological entity. Alternatively, the sampling date/time stamp may correspond to the date on which the (isolate of the) biological entity was sampled. If the database provides a sampling date/time stamp for a biological sequence, this date may be collected together with the biological sequence data. If the actual sampling date of a biological sequence is not available, a sampling date may be estimated.

In other embodiments, only partial information on sampling dates for biological sequences may be available. For instance, the sampling date may be specified with a resolution of a certain time span, such as months or years. Hence, in these embodiments, the sampling date/time stamp associated with a biological sequence may correspond to a time span. As an example, but not a limitation, for each isolate the day, month and year of the isolate and its corresponding sequence may be known. It is, however, sufficient that the year or both, the season (e.g. winter season) and the year, is specified for each isolate. In those instances where only the year of the isolate is known, the date may be set to the season in which the entity is predominantly active, e.g. if it is an influenza A isolate from the northern hemisphere, the date is set to the winter season of the year of the isolate. The winter season is defined as ranging from 1^{st} October to 31^{st} March for the northern hemisphere and from 1^{st} April to 30^{th} September for the southern hemisphere.

Furthermore, each isolate may be attributed with its geographic origin (e.g. the place of the isolation of the sequence, or preferably the place where the individual from which the sequence was isolated was infected with the fast-evolving entity). The isolates can be classified as belonging to the northern hemisphere, the southern hemisphere or as being of tropical origin. The tropical area is defined as being centered on the equator and limited in latitude in the northern hemisphere, at 23°26′ (23.4°) N latitude, and in the southern hemisphere at 23°26′ (23.4°) S latitude. The geographic regions outside the tropics belong to the northern and southern hemisphere. However, any other suitable geographic classification (e.g. by country or continent) may be used for the attribution of localization data.

At 110, the biological sequences derived from the data collected at 105 may be aligned. In bioinformatics, sequence alignment is a way of arranging the primary sequences of DNA, RNA, or protein to identify regions of identity and/or similarity. The alignment may be performed with any common alignment technique. In an embodiment, pairwise alignment techniques are used to find the best-matching piecewise (local) or global alignments of two biological sequences. As an example, but not a limitation, conventional pairwise alignment techniques such as dot-matrix techniques, dynamic programming and word techniques may be used. According to alternative embodiments, multiple sequence alignment techniques may be used. A multiple sequence alignment is a sequence alignment of three or more biological sequences, generally protein, DNA, or RNA. Multiple alignment techniques align all of the biological sequences under consideration. In some embodiments, both pairwise and multiple sequence alignment techniques may be used. Multiple alignment software is known in the art. For example, ClustalW (Higgins, D. G., Bleasby, A. J. and Fuchs, R. 1992 CLUSTAL V: improved software for multiple sequence alignment. Computer Applications in the Biosciences, volume 8, number 2, pages 189-191) can be used to generate a multiple alignment. The underlying algorithms for establishing multiple sequence alignments are known in the art and can be based on e.g. dynamic programming techniques, clustering methods, Hidden Markov model-based techniques and methods that use sequence patterns or conserved motifs to anchor parts of the multiple alignment. By way of example and not a limitation, ClustalW was used. However, any program or algorithm can be used as long as it is capable of establishing a multiple sequence alignment from biological sequences.

In typical usage, protein alignments use a substitution matrix to assign scores to amino-acid matches or mismatches, and a gap penalty for matching an amino acid in one sequence to a gap in the other. DNA and RNA alignments may use a scoring matrix, but in practice often simply assign a positive match score, a negative mismatch score, and a negative gap penalty.

After having aligned the sequences under consideration, such as the sequences collected at step 105, a distance matrix for the biological sequences may be computed at 115. Distance matrices rely on a measure of the distance between biological sequences. Distance may be defined as the fraction of mismatches at aligned positions, with gaps being ignored, counted as mismatches or otherwise weighted. In embodiments of the invention, the distance matrix may be computed using exact matching without distance correction and no weights for gaps. However, any other technique for computing a distance matrix may be used.

In more detail, a distance matrix is a matrix (two-dimensional array) containing the distances, taken pairwise, of a set of points. In this case a distance matrix can be a similarity matrix. Nucleotide similarity matrices are used to align nucleic acid sequences. For example, a simple matrix will assign identical bases a score of +1 and non-identical bases a score of −1. A more complicated matrix may give a higher score to transitions (changes from a pyrimidine such as C or T to another pyrimidine, or from a purine such as A or G to another purine) than to transversions (from a pyrimidine to a purine or vice versa). The match/mismatch ratio of the matrix sets the target evolutionary distance (States et al. 1991 METHODS—A companion to Methods in Enzymology 3:66-70); the +1/−3 DNA matrix used by BLASTN is best suited for finding matches between sequences that are 99% identical; a +1/−1 (or +4/−4) matrix is much more sensitive as it is optimal for matches between sequences that are about 70% identical. For protein sequences a PAM series of matrices may be used. PAM matrices are labeled based on how many nucleotide changes have occurred, per 100 amino acids. PAM matrices are most useful at short evolutionary distances (PAM10-PAM120). At long evolutionary distances, for example PAM250 or at only 20% identity, BLOSUM matrices are more effective.

In some embodiments, a computed distance matrix for a set of biological sequences may be obtained from a database instead of actually computing the distance matrix from raw sequence data. In those embodiments, steps 105, 110 and 115 may be omitted. The distance matrices stored in the database may have been computed in accordance with the computation of step 115. In still other embodiments, only distances needed for the subsequent steps of method 100 may be obtained from a database. In those embodiments, steps 105 (at least partially), 110 and 115 may also be omitted.

In other embodiments, data regarding an aligned set of biological sequences may be obtained from a database, such that steps 105 (at least partially) and 110 may be omitted. The alignment of the sequences stored in the database may have been done in accordance with step 110.

In embodiments of the invention, the distance matrix may be obtained from a number of different sources, including measured distance (for example from immunological studies) or morphometric analysis, various pairwise distance formulae (such as Euclidean distance) applied to discrete morphological characters, or genetic distance from sequence, restriction fragment, or allozyme data. Further, raw distance values may be calculated by simply counting the number of pairwise differences in character states of the sequences (Manhattan distance).

At step 120, a graph representation of the biological sequence data is generated based on the sampling dates/time stamps associated with the biological sequences and distances between the biological sequences. In embodiments of the invention, the distances may be derived from a distance matrix, which was either computed in step 115 or otherwise obtained.

In some embodiments, step 120 may have an initializing step, where for each set of identical sequences, all but one instance associated with the earliest sampling date/time stamp may be set aside before generating the graph representation. The retained sequence may also be the sequence associated with the latest sampling date/time stamp or a sequence chosen by any other criterion. This results in a sequence set without any duplicated sequences. Thus, in the initializing step, identical biological sequences in the set of biological sequences of interest may be determined. Then for each set of identical sequences, the biological sequence from the set of identical sequences associated with the earliest (or latest or selected according to any other criterion) sampling date/time stamp may be selected for further processing at step 120. In addition, all unique biological sequences may be selected for step 120.

In the context of the present invention, the graph representation may be a directed graph. A directed graph is a set of nodes connected by directed edges. Thus, a graph G is an ordered pair G:=(V,E), where V is a set whose elements are called nodes and E is a set of ordered pairs of nodes called directed edges.

In the present invention, let V={v₁, . . . , vₙ} be the set of nodes representing the input sequences, e.g. the sequences collected at step 105. Each node vᵢ represents a biological sequence which is associated with a specific sampling date/time stamp tᵢ such that T={t₁, . . . , tₙ} is the set of sampling dates/time stamps. In some embodiments, the sequence associated with the earliest sampling date/time stamp may be identified and the node representing this sequence may be selected as root node. Then, for each node, outgoing edges are added to all nodes with a later sampling date/time stamp resulting in the set of edges E={(vᵢ,vⱼ):tᵢ<tᵢ<tⱼ}. Accordingly, an outgoing edge from a first node to a second node may be added, if the sampling date/time stamp associated with the biological sequence represented by the second node is later than the sampling date/time stamp associated with the biological sequence represented by the first node.

In the context of the present invention, an outgoing edge of a node vᵢ is a directed edge connecting the node vᵢ with another node and being directed from the node vᵢ to the other node. Similarly, an incoming edge of a node vᵢ is a directed edge connecting the node vᵢ; with another node and being directed from the other node to the node vᵢ.

The Directed Acyclic Graph (DAG) generated in step 120 has at least (n−1) edges, if all sequences except the sequence being represented by the root node are associated with the same sampling date/time stamp. If every sequence is associated with a different sampling date/time stamp, the DAG generated during step 120 has

$$\frac{n \cdot ( n - 1 )}{2}$$

edges.

A DAG is a directed graph with no directed cycles, i.e. for any node vᵢ, there is no non-empty directed path that starts and ends on vᵢ. A path in a graph is a sequence of nodes such that from each node there is an edge to the next node in the sequence. The first node is called the start node and the last node is called the end node. Hence, a cycle is a path such that the start node and end node are the same.

To generate a weighted DAG, the edges of the graph computed in 120 are weighted. In embodiments of the invention, the edge weight of an edge may correspond to the distance between the biological sequences represented by the nodes connected by this edge. The distance between the biological sequences may be derived from the obtained/computed distance matrix or from any other source providing a distance for the biological sequences. In some embodiments, the distance between two biological sequences may be retrieved, e.g. from a public database or any other source, when an edge between nodes representing these two biological sequences is added or weighted.

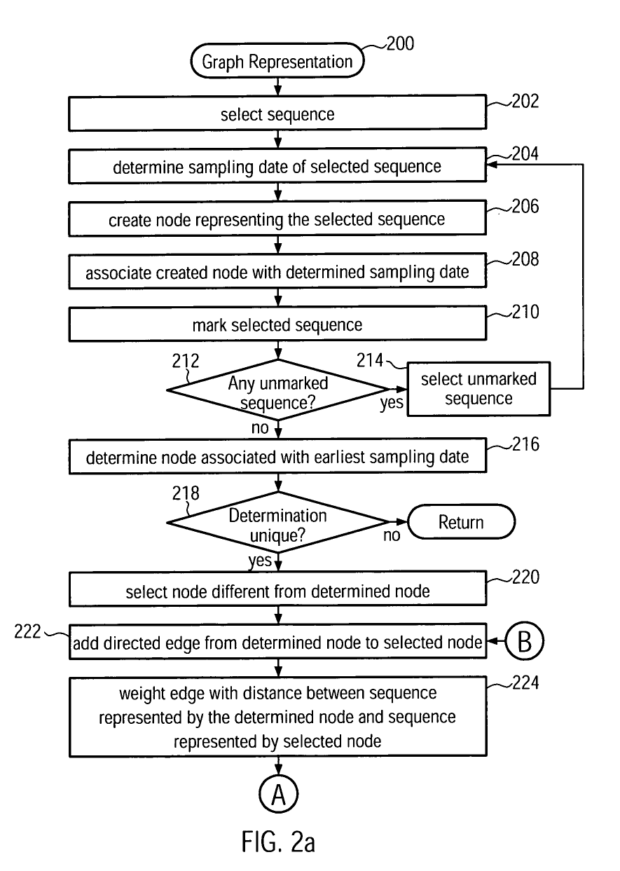

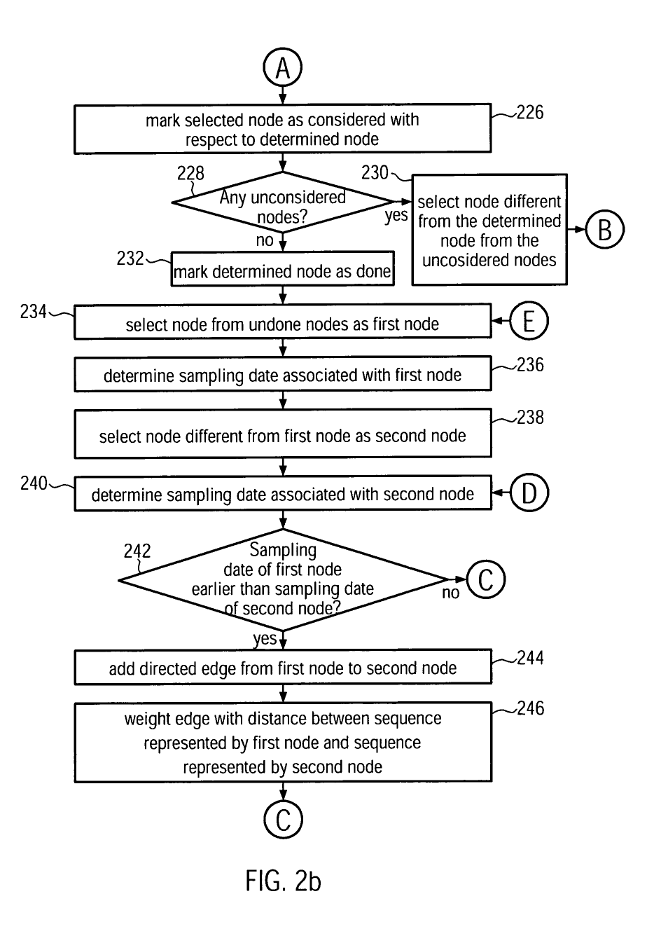

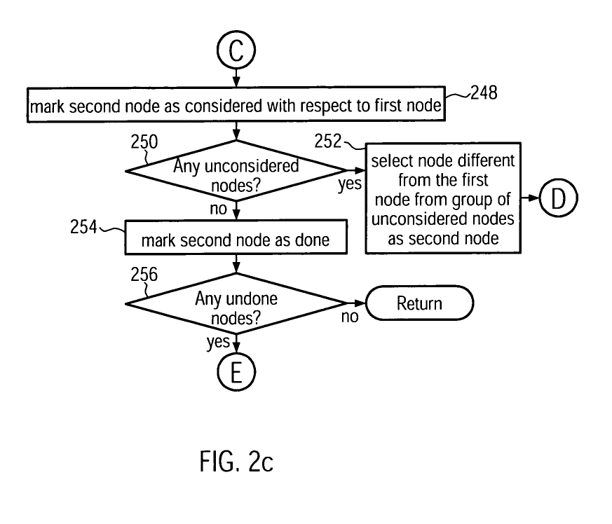

The details of the computation of the weighted DAG representing the biological sequences under consideration (step 120 of FIG. 1) will be described in more detail with respect to FIGS. 2a to 2c.

At 125, a minimum spanning tree (MST), also referred to as optimum branching, in the graph computed at step 120 is identified. A tree is a graph in which any two nodes are connected by exactly one path. Thus, a tree with n nodes has (n−1) edges. A spanning tree of a connected graph is a subgraph which is a tree and connects all the nodes of the graph. A single graph may have many different spanning trees. If a weight is assigned to each edge of the graph, these weights may be used to assign a weight to a spanning tree. As an example, but not a limitation, the weight of a spanning tree may be the sum of the weights of all the edges forming the spanning tree. An optimum branching or minimum spanning tree or minimum weight spanning tree of a directed graph may be a spanning tree with weight less than (or equal) to the weight of any other spanning tree in this graph.

There are several algorithms for identification of a minimum spanning tree in a weighted directed graph in quadratic time such as the Chu-Liu/Edmonds algorithm. However, the minimum spanning tree identified in step 125 may be computed with any available algorithm including, but not limited to the Chu-Liu/Edmonds algorithm or Bock's algorithm

According to an embodiment of the invention, a minimum spanning tree of the graph generated at step 120 is constructed by selecting for each node in the graph the incoming edge with the lowest assigned weight (distance). In case of multiple incoming edges having assigned the lowest weight, the incoming edge starting from the node representing the biological sequence associated with the latest sampling date/time stamp may be chosen from the group of edges having the lowest weight. Alternatively, in case of multiple incoming edges having been assigned the lowest weight, the incoming edge whose source node represents the biological sequence associated with the earliest sampling date/time stamp may be selected from the group of edges having the lowest weight. In still other embodiments, in case of multiple incoming edges having been assigned the lowest weight, the incoming edge may be selected randomly from the group of edges having the lowest weight. Unselected edges may be removed from the graph.

As the graph generated at step 120 is acyclic, no cycles need to be resolved.

In some embodiments, the resulting minimum spanning tree may be selected as the phylogenetic tree for the set of biological sequences under consideration. Thus, a phylogenetic tree constructed with the method 100 is the phylogenetic tree for the biological sequences under consideration with the least overall weight (cost) with respect to the defined distances between the biological sequences among all possible phylogenetic trees for those biological sequences. In contrast to conventional techniques for constructing phylogenetic trees, the method 100 results in phylogenetic trees that may be multifurcating instead of bifurcating.

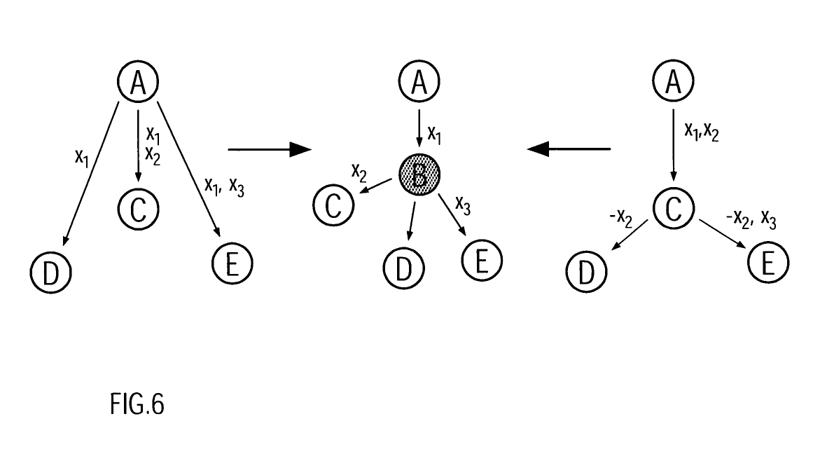

In some embodiments, the resulting minimum spanning tree from method 100 may be an initial optimum branching. In an update step, sequences of unsampled evolutionary intermediates are inferred based on differences between sequences represented by nodes connected by an edge of the initial minimum spanning tree. Then, time stamps are assigned to the inferred unsampled evolutionary intermediates (the details on inferring unsampled evolutionary intermediates and assigning time stamps to the inferred intermediates will be discussed below with respect to FIG. 6). The inferred sequences are added with assigned time stamps to the sequence set under consideration and steps 120 and 125 as described above may be performed for this set of sequences. This update step may be repeated until no new intermediates are found (inferred).

For embodiments where step 120 comprises the initializing step, the sequences not selected at each of steps 120 (the update step is an iterative process repeating steps 120 and 125) are added to the sequence set derived in the update step, if no new evolutionary intermediates are found. From this plurality of sequences an optimum branching may be computed according to steps 120 and 125 and the resulting minimum spanning tree may be identified as the phylogenetic tree.

According to embodiments of the invention, the method 100 may also be used if only partial information on sampling dates is available, i.e. if the sampling date is only specified with a resolution of a certain time span such as months or years. In this case, all nodes representing biological sequences associated with sampling dates/time stamps within the same time span may be connected in the graph generated during step 120. However, the graph resulting from this computation is not an acyclic graph. Therefore, cycles in the graph are resolved by computation of an minimum spanning tree from weighted directed graphs as described in R. E. Tarjan, Networks, volume 7, pages 25-35. This may be done at step 125 during the computation of the minimum spanning tree.

According to embodiments of the invention, the method 100 may also be used if no information on the sampling dates is available. In this case, all nodes representing biological sequences are connected with undirected edges to each other and a least cost tree or minimum spanning tree may be computed from the resulting weighted undirected graph using a minimum spanning tree algorithm for undirected graphs such as Prim's algorithm, Kruskal's algorithm, Bor [symbol] vka's algorithm, the reverse-delete algorithm or the algorithm developed by Bernard Chazelle. This may be done at step 125 during the computation of the minimum spanning tree.

Referring now to FIGS. 2a to 2c, details of the graph generation step 120 of method 100 according to some embodiments of the invention are described. At 202, data representing a biological sequence is selected. In some embodiments, this selection may be done randomly, while in other embodiments, the biological sequence associated with the earliest/latest sampling date/time stamp may be selected. If the sequence was selected randomly, the associated sampling date/time stamp is determined at step 204. In embodiments where the sequence is selected according to its sampling date/time stamp, the sampling date/time stamp may already be determined at step 202.

At 206 a node is created representing the selected biological sequence. This node is associated with the determined sampling date/time stamp of the selected sequence at 208. The so processed biological sequence data may be marked as being represented by a node at 210.

At 212 it is determined whether any unmarked biological sequence data remains in the set of biological sequences under consideration. If there is unmarked biological sequence data, unmarked biological sequence data representing a biological sequence not yet represented by a node is selected at 214 and the method branches back to step 204.

However, if it is determined at 212 that there is no unmarked biological sequence, i.e. all biological sequences in the set of biological sequences of interest are represented by a node, the method 200 branches to step 216, where the node representing the biological sequence data associated with the earliest sampling date/time stamp is determined.

At 218, it is determined whether the determination performed in step 216 results in a unique node. If the determination results are not unique, the method may abort and return an error. In alternative embodiments, a node may be randomly selected from the group of nodes representing biological sequences sharing the earliest sampling date/time stamp while the other nodes in the group of nodes representing biological sequences sharing the earliest sampling date/time stamp may be left unconsidered in the following steps 220 to 256. In some embodiments, each node from the group of nodes representing biological sequences sharing the earliest sampling date/time stamp may be selected and for each selected node, the following method steps 220 to 256 may be performed, wherein the respective unselected nodes in the group of nodes representing biological sequences sharing the earliest sampling date/time stamp may be left unconsidered. In those embodiments, as many graph representations as nodes in the group of nodes representing biological sequences sharing the earliest sampling date/time stamp may be generated.

If it is determined at 218 that the determination of 216 results in a unique node, a node different from the node determined at 216 is selected at 220. In cases, where the results of the determination at 216 were not unique and a node was selected randomly at 218, this randomly selected node corresponds to the determined node.

At 222 a directed edge (outgoing edge with the determined node as source node and the first node as sink node) is added from the determined node to the selected node. At 224 the distance between the biological sequences represented by the determined node and the selected node is assigned to the added directed edge as edge weight. The distance between the biological sequences may be derived from the obtained/computed distance matrix (as, for instance, described with respect to step 115 of FIG. 1) or from any other source providing a distance for these biological sequences.

At 226 the selected node may be marked as considered with respect to the determined node. At 228 it is determined whether there is any node that is not considered with respect to the determined node, i.e. not marked as considered with respect to the determined node. If it is determined at 228 that there are unconsidered nodes, the method proceeds to 230, where a further node different from the node determined at 216 is selected from the group of nodes that are not marked as considered. Then the method branches back to step 222.

If it is, however, decided at 228 that all nodes have been considered with respect to the node determined at 216, the method proceeds to 232 and marks the determined node as done.

At 234 a node from the group of undone nodes is selected as first node. This selection may be random or under consideration of the sampling date/time stamp associated with sequences represented by the nodes, i.e. the earliest sampling date/time stamp associated with the biological sequences represented by nodes in the group of undone nodes may be identified and a node representing a biological sequence associated with this earliest sampling date/time stamp may be selected as first node. If the first node was randomly selected at 234, the sampling date/time stamp associated with the biological sequence represented by that node may be determined at 236. In embodiments where the first node is selected under consideration of the sampling date/time stamp, the sampling date/time stamp of the biological sequence represented by the first node may be determined at step 234.

At 238 a node different from the first node and different from the node determined at 216 is selected as second node. In embodiments, where the first node was selected under consideration of the sampling date/time stamp, i.e. the earliest sampling date/time stamp associated with the biological sequences represented by nodes in the group of undone nodes was identified and a node representing a biological sequence associated with this earliest sampling date/time stamp was selected as first node at 234, the second node may be selected from the group of undone nodes, wherein the group of undone nodes does not include the first node such that the second node is different from the first node. At 240 the sampling date/time stamp associated with the biological sequence represented by the second node is determined and compared at 242 with the sampling date/time stamp associated with the biological sequence represented by the first node.

If the sampling date/time stamp associated with the biological sequence represented by the first node is not earlier than the sampling date/time stamp of the biological sequence represented by the second node, the method branches from 242 to step 248.

However, if the sampling date/time stamp associated with the biological sequence represented by the first node is earlier than the sampling date/time stamp of the biological sequence represented by the second node, the method branches from 242 to step 244, at which an outgoing edge is added from the first node to the second node. This newly added edge is weighted at 246 with the distance between the biological sequences represented by the first and second node. The distance between the biological sequences may be derived from the obtained/computed distance matrix (as, for instance, described with respect to step 115 of FIG. 1) or from any other source providing a distance for these biological sequences.

At 248 the second node is marked as considered with respect to the first node. At 250 it is decided whether there are any nodes not considered with respect to the first node, i.e. not marked as considered with respect to the first node.

If there are unconsidered nodes with respect to the first node, a node different from the node determined to represent the biological sequence being associated with the earliest sampling date/time stamp (step 216) and the first node is selected from the group of nodes not marked as considered with respect to the first node as the second node at 252. Then the method branches back to step 240.

In embodiments, where the first node was selected under consideration of the sampling date/time stamp at 234, i.e. the earliest sampling date/time stamp associated with the biological sequences represented by nodes in the group of undone nodes was identified and a node representing a biological sequence associated with this earliest sampling date/time stamp was selected as first node at 234, the second node may be selected from the group of undone, unconsidered nodes not including the first node such that the second node is different from the first node.

If all nodes have been considered with respect to the first node, the first node is marked as done at 254. At 256 it is decided whether all created nodes are done. If there is any undone node, the method branches back to 234. However, if all nodes are done, the method returns.

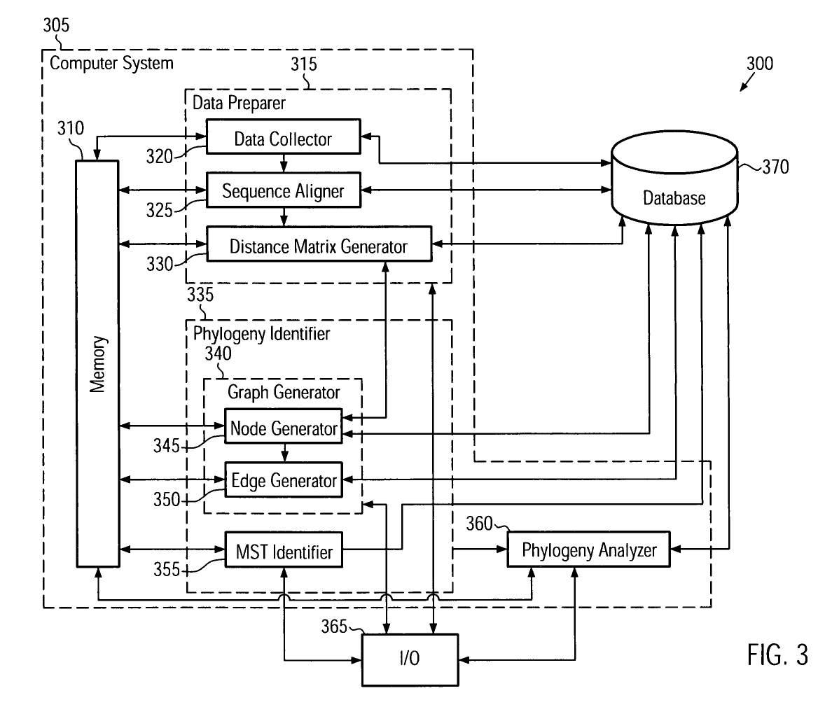

Turning now to FIG. 3, an exemplary system 305 adapted to carry out the methods described with respect to FIGS. 1 and 2 is described. The system 305 may be a general purpose computer system such as a mainframe computer, a workstation, a personal computer, a laptop or any other suitable computing device. In an embodiment of the present invention the computer system 305 may comprise a memory 310, a data preparer 315, a phylogeny identifier 335 and optionally, a phylogeny analyzer 360. The memory 310, the data preparer 315, the phylogeny identifier 335 and the phylogeny analyzer 360 may be communicatively coupled to each other, for instance via an internal bus system, for data exchange. In addition, the data preparer 315, the phylogeny identifier 335 and the phylogeny analyzer 360 may output data via an interface to external output devices 365 such as a display or a plotter. The modules 315, 335 and 360 may also receive input via an interface from external input devices 365 such as a keyboard or a mouse. In some embodiments, the modules 315, 335 and 360 may additionally or alternatively store their outputs to or receive their inputs from a computer-readable medium, accessible via an input/output device 365. In some embodiments, the memory 310 may also be couple to input/output (I/O) devices 365 to communicate with those device. In those embodiments, the modules 315, 335 and 360 may also output data to and receive instructions from the I/O devices 365 via the memory 310.

In addition, the computer system 315 may be coupled to an external database 370, for instance, via the internet or any other network. In those embodiments, each of the modules 315, 335 and 360 may retrieve input data from the database and/or transmit results to the database 370. Alternatively or in addition, the modules 315, 335 and 360 may communicate with the database 370 via memory 310 which is also coupled to the database 370 in those embodiments.

The database 370 may store results from modules 315, 335 and 360 for later retrieval for computer systems similar to system 305 that are coupled to the database 370. Additionally, the database may store data needed for the computations carried out on computer system 305, such as biological sequences, sampling dates or time stamps associated with the biological sequences, sets of aligned biological sequences, distances between biological sequences, distance matrices for sets of biological sequences, graphs for a set of biological sequences generated in accordance with step 120 of FIG. 1 and/or method 200 of FIGS. 2a to 2c, and phylogenetic trees built in accordance with method 100.

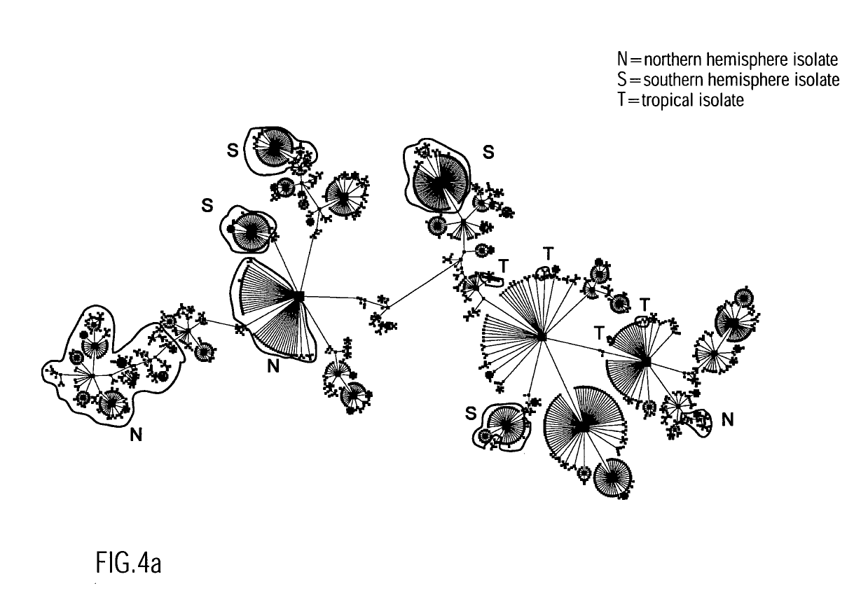

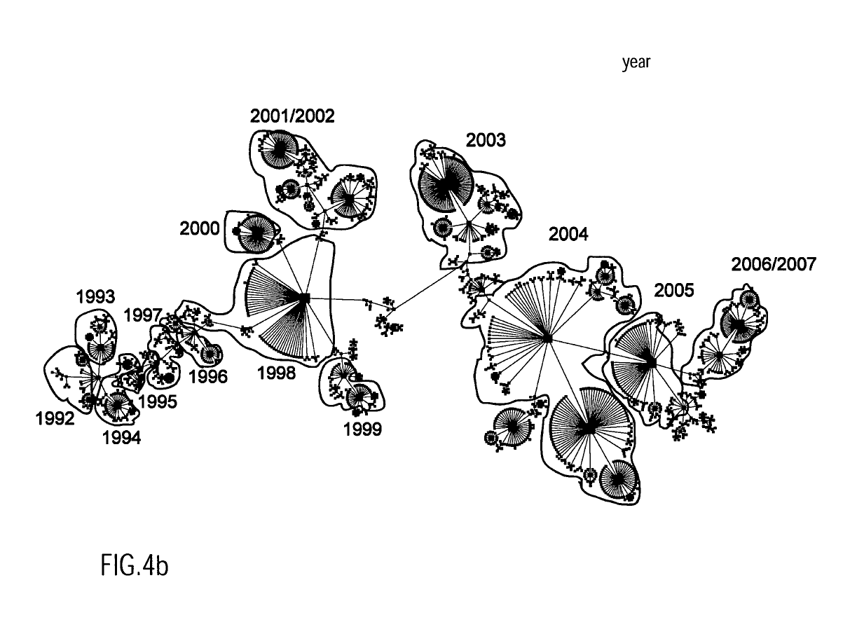

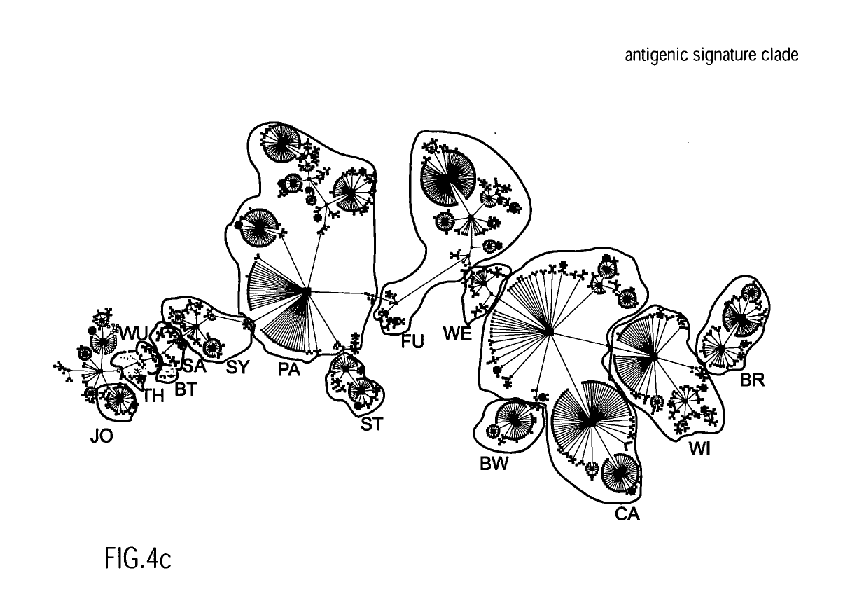

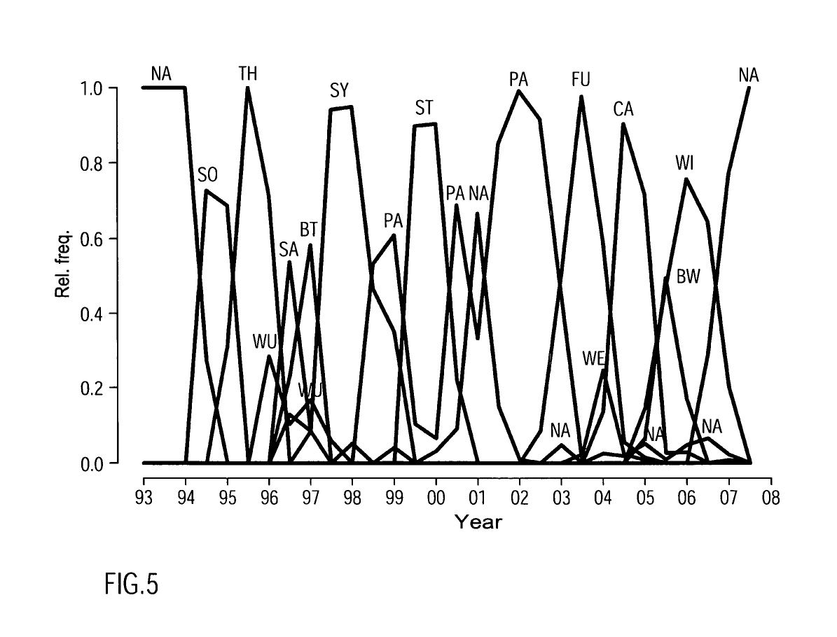

In embodiments of the invention, the data preparer 315 may be adapted to carry out steps 105 to 115 of FIG. 1 and the phylogeny identifier 335 may be adapted to carry out steps 120 and 125 of FIG. 1 as well as method 200 of FIGS. 2a to 2c. In some embodiments, the computer system 305 may additionally comprise the phylogeny analyzer 360, which is adapted to interpret the built phylogenetic trees as described below with respect to FIGS. 4 to 5.

The data preparer 315 may comprise a data collector 320, a sequence aligner 325 and a distance matrix generator 330. The data collector 320 may be adapted to perform step 105 of method 100 and thus may be coupled to the database 370 to retrieve data needed by the sequence aligner 325, the distance matrix generator 330, the phylogeny identifier 335 and/or the phylogeny analyzer 360. The data collector 320 may directly provide modules 325, 330, 335 and/or 360 with the collected data or indirectly via the memory 310. The sequence aligner 325 may be adapted to perform step 110 of method 100. To that end, it may retrieve biological sequence data directly from the external database 370 or from the data collector 320.

The distance matrix generator 330 may be adapted to perform step 115 of method 100. In some embodiments, module 330 may compute distances between aligned sequences provided by the sequence aligner 325 or by the external database 370 and based on these distances generate a distance matrix. In other embodiments, the distance matrix generator 330 may additionally or alternatively retrieve distances between biological sequences from external database 370. In still other embodiments, the data collector 320 may retrieve a distance matrix for a set of biological sequences from the database 370 or retrieve distances between biological sequences from external database 370 on a request from the phylogeny identifier 335 without actually providing a complete distance matrix. In those embodiments, the sequence aligner 325 and/or the distance matrix generator 330 may be inactive.

The data preparer 315 outputs a distance matrix or distances between biological sequences to the phylogeny identifier 335 and/or the memory 110. In some embodiments, the data preparer 315 may also transmit the computed distance matrix to the external database 370.

The phylogeny identifier 335 is adapted to retrieve data relating to distances between biological sequences and sampling dates or time stamps relating to the biological sequences from the data preparer 315, the memory 110 and/or directly from the external database 370. The phylogeny identifier 335 may comprise a graph generator 340 adapted to perform step 120 of method 100 and/or method 200, as well as an MST identifier 355 adapted to perform step 125 of method 100.

The graph generator 340 may comprise a node generator 345 adapted to perform steps 202 to 214 of method 200 and an edge generator 350 adapted to perform steps 216 to 256 of method 200. Both the node generator and the edge generator may be communicatively coupled to the memory 110 and/or the external database 370 to provide results to or retrieve data from module 110 or the database 370. In some embodiments, the graph generator 340 may be adapted to obtain graphs from the database 370. The graph generator may also be coupled to the I/O devices 365 to display or output the generated graph. Also, the graph generator 340 may retrieve data from or output data to a computer-readable medium accessible via an I/O device 365.

The minimum spanning tree identifier 355 may be coupled to the memory 310, the graph generator 340, the I/O devices 365 and/or the external database 370. The MST identifier 355 may be adapted to compute a minimum spanning tree in accordance with step 125 of method 100. To this end, the MST identifier 355 may retrieve a graph from the graph generator 340, the memory 310 or the database 370 and output the identified MST to the memory 110, the I/O devices 365, the external database 370 and/or the phylogeny analyzer 360.

The phylogeny analyzer 360 is communicatively coupled to the database 370, the phylogeny identifier 335, the memory 310 and the I/O devices 365. The phylogeny analyzer is adapted to interpret phylogenetic trees with a consistent timeline built by the phylogeny identifier 335 or retrieved from the database 370 as described in more detail below with respect to FIGS. 4 to 5.

The invention according to methods 100 and 200 may be described in the general context of computer-executable instructions, such as program modules, being executed by a computer such as computer 305. Hence the method 100 and 200 may be computer-implemented. Generally, program modules include routines, programs, objects, components, data structures, etc. that perform particular tasks or implement particular abstract data types. The general purpose or special purpose computing system environments or configurations may be programmable using a high-level computer programming language. In some embodiments, the general purpose or special purpose computing system environments or configurations may also use specially-programmed, special-purpose hardware.

The invention may also be practiced in distributed computing environments where tasks are performed by remote processing devices that are linked through a communication network. In a distributed computing environment, program modules may be located in both local and remote computer storage media including memory storage devices.

In the research of disease and drug resistance, sequences are increasingly becoming available for isolates that have been sampled at successive points in time. Embodiments of the present invention find phylogenies based on characteristic mutation patterns of unsampled evolutionary intermediates for this data. The evolution of the major surface protein and antigenic determinant of influenza A for the time period from 1993 to 2007 was studied with methods of the present invention, using all available dated sequences. The analysis of repeatedly occurring mutations in the phylogeny revealed a significant association with antigenic drift and their suitability as genetic markers of viral fitness and identification of vaccine strain candidates. The phylogeny is divided in distinct clades representing the major antigenic types of the virus for the analyzed time period. The present invention allows for studying evolution from large-scale data sets and returns multifurcating tree topologies which visualize the genetic diversity and geographic prevalence of emerging lineages.

Antigenic lineages: Between 1993 and 2007, several distinct antigenic types of H3N2 have emerged in the human population by antigenic drift of the epitope sites. The genetic changes of novel strains relative to previous reference strains are continuously monitored and reported by the influenza centers of the WHO. As antigenic types have only been reported for very few sequences, the signature mutations are used to partition the data and identify phenotype-related clades in the phylogeny. All analyzed mutation signatures (lists of mutations) for the different antigenic types of influenza subtype H3N2 during the analyzed time period (1993-2007) are given in the following table (Table 2).

TABLE 1
Mutation signatures described by the World Health Organization in
their semiannual reports for antigenic types of influenza A, subtype H3N2
between 1993 and 2007. Positions refer to the HA1 subunit of the
hemagglutinin protein of influenza A, subtype H3N2. Mutations describe
the differences of a new predominant antigenic type relative to the
previous predominant antigenic type.

| Type | Mutation signature |
|---|---|
| Johannesburg (JO)ₑ | S47P, D124N, S219Y |
| Thessaloniki (TH)_{d} | G172D, F216N, F219S, S278N |
| Wuhan (WU) | K135T, N145K, N262S |
| Bratislava (BT)ₐ | R57Q, K92T, N122K, G275D |
| South Africa (SA) | T121N/T, G124S, D133N, G142R/G |
| Sydney (SY) | K62E, K156Q, E158K, V196A, N276K |
| Panama (PA) | I144N/D, K172E, T192I |
| Stockholm (ST)_{c,e} | G5V, Q33H, K92T, N144I, D271N |
| Fujian (FU) | L25I, H75Q, H155T, Q156H/Q, A131T, V202I, |
| | G225D |
| Wellington (WE) | Y159F, S189N, S227P |
| California (CA) | Y159F, S189N, V226I, S227P |
| Baden-Wuerttemberg | V112I, N145S, Y159F, K173E, S189N, V226I, |
| (BW)_{b} | S227P |
| Wisconsin (WI) | S193F, D225N |
| Brisbane (BR) | G50E, K140I |

ₐA Wuhan-like clade which appeared in the northern hemisphere in 96/96.
_{b}A subclade of the antigenic type California appearing in the summer of 2005.
_{c}A subclade of the antigenic type Panama.
_{d}A subclade of the antigenic type Johannesburg
ₑMutations inferred from labeled strains.

FIGS. 4a to 4c shows an exemplary phylogenetic tree built using method 100 and/or method 200. A suitable computer environment for building these trees may be computer system 305 of FIG. 3.

Each of FIGS. 4a to 4c shows the same phylogeny for 1813 isolate sequences of the H3 hemagglutinin precursor gene sampled between 1993 and 2007. Nodes correspond to individual sequences. The node size reflects the number of direct descendants. Nodes are labeled by geographic sampling location (FIG. 4a), season of sampling (FIG. 4b) and antigenic clades (FIG. 4c). For the sake of clarity, merely some of the nodes of the tree are labeled. The skilled person, however, will understand that each isolate node of FIG. 4a may be assigned a geographic sampling location, each isolate node of FIG. 4b may be assigned a season of sampling and each isolate node of FIG. 4c may be assigned an antigenic signature clade. Seasons start with 1992, corresponding to the 92/93 winter season in the northern hemisphere.

An H3 phylogeny according to method 100 was constructed from 1813 dated genetic sequences of the HA0 precursor protein of influenza subtype H3N2. The data were obtained from the NCBI influenza virus resource. They comprise isolates from 35 countries and span the time period from Jan. 16, 1993 (92/93) season in northern hemisphere) to Jul. 3, 2007 (2007 season in the southern hemisphere). 60% of the sequences originate from the northern hemisphere (mostly the United States), 36% from the southern hemisphere (mostly New Zealand and Australia), and 4% from the tropics (mostly from countries in south-east Asia; 80%). Sequences were aligned with ClustalW, version 1.93 using default settings. A nucleotide distance matrix was computed in which distances reflect the number of distinct positions between each pair of sequences in the alignment with gaps being considered a fifth character.

A phylogeny corresponds to a tree built in accordance with method 100. The root node represents the isolate with the earliest sampling date/time stamp. In accordance with method 100, for each node, outgoing edges were added to all nodes with a later sampling date/time stamp.

The phylogeny with the least overall cost (weight) in genetic distance is a rooted minimum spanning tree in this graph as discussed above with respect to method 100. For the trees of FIGS. 4a to 4c, the Chu-Liu/Edmonds algorithm was used and a minimum spanning tree was constructed by selecting the incoming edge with the lowest cost (weight) for each node. In case of multiple incoming edges with identical cost, the edge with the earliest sampling date/time stamp for the associated source node was chosen. As the input graph is acyclic, no cycles need to be resolved.

The resulting phylogeny of H3 hemagglutinin evolution for the time period from 1993 to 2007 is shown in FIGS. 4a to 4c.

As also described with respect to FIG. 1, a phylogenetic tree may be inferred as follows:

The input to the method is a plurality of sequences with associated sampling dates/time stamps and isolate identifiers.

In an initializing step, for each set of identical sequences among the input sequences, all but one sequence corresponding to the one with the earliest sampling date/time stamp are set aside. The retained sequence may also be the sequence with the latest sampling date/time stamp or a sequence chosen by any other criterion from a set of identical sequences. This results in a sequence set without any duplicated sequences. For the plurality of non-identical sequences an initial optimum branching is computed.

In an update step, the sequences of unsampled evolutionary intermediates are inferred based on differences in sequence between nodes connected by an edge in the optimum branching. The inferred sequences are then added with time stamps to the sequence set. Then, for sets of identical sequences, all but one instance, which may be the instance with the earliest time stamp, are removed. Then, an optimum branching is recomputed. This step is repeated until no new intermediates are found.

In a final step, the sequences that have been set aside in the initializing step are added to the sequence set derived in the update step. From this plurality of sequences an optimum branching may be computed and may be identified as the phylogenetic tree.

Unsampled evolutionary intermediates can be inferred from sequence differences between the sequences associated with the nodes in the minimum spanning tree. FIG. 6 shows exemplary how to infer a phylogeny and the sequences of unsampled evolutionary intermediates from sequence differences in the minimum spanning tree. The middle panel represents the true phylogeny for five isolates. The associated mutation events x₁ to x₃ are shown on the edges of the tree. As an example, x₁ may represent a difference at position 183 between the source (A at position 183 for the source) and sink isolate sequences (G at position 183 for the sink), denoted by A183G. −x₁ in this case denotes the reverse of the change, G183A, with G at position 183 of the source node sequence and A at position 183 of the sink node sequence. In the absence of a sequence representing the evolutionary intermediate B among a plurality of analyzed sequences, a minimum spanning tree of the sampled sequences A, C, D and E is represented by the tree topologies on the left and right side of the panel. The associated mutations x₁ to x₃ are shown on the edges of the trees. Which of these tree topologies is obtained depends on how the least-cost incoming edge is selected in case of multiple incoming least cost edges for a node. In this exemplary case, the choice of the edge may be random, based on minimum or maximum difference in time between the associated sampling dates/time stamps or in some other fashion. Given one of the shown topologies, the intermediate sequence B can be inferred from the mutation patterns on the in- and/or outgoing edges of a node as follows:

In the event of sets of mutations occurring repeatedly on more than one outgoing edge of a node (left part of figure), these are used to infer the sequences of unsampled intermediates by the following procedure: In the example on the left, there is one set which contains mutation x₁. If multiple sets exist, for each pair of mutation sets Xᵢ and Xⱼ, it is determined whether the relative complements Xᵢ\Xⱼ and Xⱼ\Xᵢ are empty. If for any pair of mutation sets both relative complements are non empty, the node is flagged as “ambiguous”. For ambiguous nodes, intermediates are processed in the inference step when no unambiguous nodes remain. Then, an intermediate sequence is created by introducing the changes specified by the largest mutation set into the sequence associated with the parent node.

If a node is not flagged ambiguous, for each mutation set, an intermediate sequence is created. This may be done by introducing the changes specified by the mutation set into a copy of the sequence associated with the parent node. In the example on the left, an intermediate sequence may be derived by introducing mutation x₁ into a copy of the sequence of node A. Alternatively, the intermediate sequence may also be derived from the sequence of a descendant node such as C by introducing the reverse of all mutations occurring on the edge of isolate A to C and not contained in the mutation set. The intermediate is then added to the sequence set with a sampling date/time stamp. The sampling date/time stamp is chosen from the time span after the sampling date/time stamp of node A and before the sampling dates/time stamps of the descendant nodes (C, D and E in the example on the left). If a mutation set specifies a subset of the mutations contained in one or more other sets, the sampling date/time stamp for the inferred sequence is chosen from the time span after the sampling dates/time stamps of sequences inferred from these other sets and before the sampling dates/time stamps of the descendant nodes.

The second mutation pattern which may be used to infer the sequence of an unsampled intermediate is a set of (one or more) mutations which occur in reverse order on the in- and outgoing edges of a node, as shown for node C in the example on the right. In this case, the unsampled sequence of the intermediate B may be created from a copy of the sequence associated with node A by introducing all changes on the outgoing edge of A to C which are not reversed on the outgoing edges of C. Alternatively, the sequence associated with node B may be created from a copy of the sequence associated with node C by introducing the reverse of the set of mutations. The new intermediate sequence B is added with a time stamp from the time span after the sampling date/time stamp of node A and before the sampling date/time stamp of node C to the set of sequences.

In the case that different sets of mutations are reversed on different outgoing edges of a node relative to the incoming one, it is first checked for each pair of mutation sets whether the relative complement of both sets is non-empty. If this is case, the node is masked (no intermediates are created based on the patterns at this node) until the remainder of the tree is stable and then an intermediate sequence is created by introducing the changes specified by the largest set of reversed mutations. If this is not the case, for each set of mutations an intermediate sequence is created as described above. The new intermediate sequences are added with time stamps from the time span after the sampling date/time stamp of node A and before the sampling date/time stamp of node C to the set of sequences, in an order according to the number of introduced mutations, such that the intermediate with the maximum number of introduced changes is assigned the earliest time stamp and that no intermediate with a given number of introduced changes has a later time stamp than another intermediate with less changes introduced.

TABLE 2
Comparison of tree cost (# mutations) and runtime for trees inferred
using mutation patterns (PPhylo) and bifurcating maximum parsimony trees
computed with PAUP for 13 different sequence sets for influenza subtype H3N2
obtained from the Influenza Sequence Database at NCBI, the number of
sequences in a particular set is given in brackets. HA0 refers to the complete
sequence of the hemagglutinin precursor, HA1 to subunit 1 of the protein.
Sequence sets 3-13 represent sets of all other viral proteins of the virus with the
exception of PB1-F2. Runtimes are given in seconds.

| | PPhylo | PAUP | PPhylo | PPhylo | PAUP |
|---|---|---|---|---|---|
| Data set (# sequences) | cost | cost | Extra cost (%) | Runtime (s) | Runtime (s) |
| 1. HA0, year of 1993 (38) | 87 | 87 | 0.00% | 12 | 0.18 |
| 2. HA1, 1993-2007 (1813) | 1913 | 1897 | 0.84% | 6749 | 421755 |
| 3. HA1, 1993-2007 (3142) | 3737 | 3705 | 0.86% | 33638 | 4148239 |
| 4. HA0, 1993-2007 (1366) | 2180 | 2166 | 0.65% | 9163 | 195611 |
| 3. NA, 1993-2007 (1366) | 2013 | 1989 | 1.21% | 4209 | 248995 |
| 6. M1, 1993-2007 (1366) | 538 | 535 | 0.56% | 400 | 416706 |
| 7. M2, 1993-2007 (1366) | 400 | 390 | 2.56% | 84 | 1055683 |
| 8. NP, 1993-2007 (1366) | 1512 | 1494 | 1.20% | 5292 | 175742 |
| 9. NS1, 1993-2007 (1366) | 662 | 655 | 1.07% | 561 | 495710 |
| 10. NS2, 1993-2007 (1366) | 354 | 345 | 2.61% | 115 | 1474474 |
| 11. PA, 1993-2007 (1366) | 2131 | 2115 | 0.76% | 9627 | 122390 |
| 12. PB1, 1993-2007 (1366) | 2375 | 2362 | 0.55% | 13309 | 86257 |
| 13. PB2, 1993-2007 (1366) | 2271 | 2254 | 0.75% | 9268 | 100706 |

The cost in number of mutations and run time on a Dell Poweredge 6950 (4× Opteron 8220 2.8 GHz 2×1 MB, Dual Core AMD Opteron Processor) for phylogenetic trees computed with the described method with thirteen sequence sets of different proteins of influenza, subtype H3N2 were compare to maximum parsimony trees computed with PAUP (version 4.0b10 for UNIX). PAUP* was used with the default settings except the following options. The sequences were handled as datatype=‘STANDARD’ to disable usual DNA macros. Gaps were treated as additional state (GAPMODE=NEWSTATE), i.e. as a fifth base. The trees were rooted with the oldest sequence. The maximum number of stored trees was set to 200 and was not further increased. The optimization criterion was set to ‘parsimony’ and the tree space was searched by heuristic search, where the sequences were randomly added within the tree building procedure for the initial tree. Table 2 shows the mutation cost and runtimes for the different data sets; the described method is 10-1000 times faster and generates trees with an extra cost of 0-2.61% in terms of mutations.

The phylogenetic tree can then be used to analyze the development of antigenic lineages (FIG. 4c), to identify sites under positive selection, amino acid mutations associated with antigenic drift and viral lineages with the potential to become predominant in the next winter season, as well as the geographic spread and origin of successful strains (FIG. 4a).

Antigenic lineages: For visualizing the antigenic lineages or antigenic clades, in a first step, the node sequences are assigned based on the presence of signature mutations (Table 1). If one isolate matches multiple signatures, the isolate is assigned to the latest occurring type (e.g. isolates of the type Panama can still carry the signature mutations of the previous type Sydney, as the characteristic positions are distinct). If a descendant of a signature-matching isolate acquired a change in one of the signature sites that does not match a subsequently appearing signature, it is left unassigned. In the example analysis 92% (1673) of the isolates could thus be assigned.

Visualization of the antigenic assignments reveals a consistent clade structure (FIG. 4c). Isolates in a clade are antigenically similar but not necessarily identical. According to WHO reports, in particular in the late stages of circulating types, variation in antigenicity is observed. To elucidate phenotypic properties in detail, experimental tests of viral isolates with techniques such as the hemagglutination inhibition assay can be performed, and the similarity can be revealed with known techniques such as multidimensional scaling [F. J. Poelwijk, et al., Nature 445 (7126), 383 (2007)]

For all clades, the timing of prevalence shown in the tree of FIGS. 4a to 4c agrees with epidemiological reports (FIG. 5). Types (named after vaccine strains) appear as predominant in the following order:

Johannesburg (1994-1995) and the Johannesburg-like clade Thessaloniki (1995-1996), Wuhan and the Wuhan-like clade Bratislava (1996-1997), Sydney (1997-1998), Panama (1998-2003) and the clade Stockholm (1999-2000), Fujian (2002-2004), California (2004-2005) and the California-like clade Baden-Wuerttemberg (2005-2006), Wisconsin (2005-2007) and Brisbane (clade I-3 of Wisconsin, 2006-2007). The reported signature mutations can therefore reveal the antigenic clade structure of the tree and allow to study their properties in the phylogeny.

Sites under positive selection: The exemplary influenza phylogeny contains 1915 nucleotide and 926 amino acid mutations. The majority of amino acid mutations occur at the tips of the tree, 718 nucleotide and 344 amino acid changes are located on internal branches. Changes at hemagglutinin sites under positive selection possess predictive value for the identification of progenitors for future (e.g. H3) lineages. Since 1999, the data has more than quadrupled, which allows the identification of novel sites of importance. These novel sites of importance are characterized by a significant excess of nonsynonymous over synonymous mutations on internal branches of the phylogeny.

A synonymous mutation is defined as a mutation in which one nucleotide is substituted for another, but the encoded amino acid remains unchanged (i.e., UUA to UUG, both specifying leucine). A nonsynonymous mutation is defined as nucleotide substitution that changes the amino acid specified (i.e., AGC to AGA, or serine to arginine). A nonsynonymous substitution is equivalent to a nonsynonymous mutation or amino acid mutation or amino acid change.

Sites under positive selection were identified as described [R. M. Bush, et al., Mol. Biol. Evol. VOL 16 NO 11, page 1457 (1999)], based on a significant excess of nonsynonymous over synonymous mutations on internal branches of the phylogeny. The probability for a nonsynonymous substitution to occur under the null model of no positive selection was calculated from the phylogeny based on the relative frequency of nonsynonymous mutations among mutations in non-epitope regions of the protein. The significance of an observed ratio of synonymous to nonsynonymous mutations on internal branches of the phylogeny at a particular site was calculated from the binomial distribution. The multiple testing error was corrected with the false discovery rate [Y. and Hochberg Benjamini, Y., Journal of the Royal Statistical Society VOL 57, NO 289 (1995)].

16 such sites under positive selection were identified in the HA1 subunit sequences (with p-values <0.05). 11 of the sites are in or close to the epitope regions of HA1, and most are exposed on the protein surface (Table 3).

TABLE 3
Sequence positions in HA1 subunit under positive selection
identified by significant enrichment of non-synonymous
substitutions on internal branches of the phylogeny.

| | | | Antigenic drift | Antigenic | Bush et al. | % Surface |
|---|---|---|---|---|---|---|
| Site | Nsyn. | Syn. | residue ^{d} | site ^{a} | site ³ | exposure ^{b} |
| 45 | 6 | 0 | | | | 13.6413 |
| 49 | 4 | 0 | | | | 8.2949 |
| 50 | 10 | 1 | | x | | 25.9763 |
| 53 | 5 | 0 | C | x | | 15.9248 |
| 121 | 4 | 0 | | x | X | 13.9794 |
| 138 | 4 | 0 | | | X | 3.5439 |
| 142 | 7 | 1 | | x | X | 18.2336 |
| 144 | 7 | 0 | A | | | 7.3392 |
| 145 | 9 | 0 | A | x | X | 16.0612 |
| 173 | 4 | 0 | | x | | 36.0493 |
| 186 | 5 | 1 | B | x | x | 5.3243 |
| 199 | 5 | 1 | | x | | 23.7655 |
| 220 | 6 | 0 | D | x | | 0 |
| 226 | 10 | 6 | X | x | X | 12.7562 |
| 229 | 6 | 1 | | | | 4.1469 |
| 275 | 8 | 1 | C | x | X | 16.8292 |

^{a} 89 sites in antigenic regions defined in [D. C. Wiley, et al., Nature 289 (5796), 373 (1981)] with more than 7% surface exposure of residues except for positions in the subunit interface region (site D).
^{b} Accessibility computed with method at http://swift.cmbi.ru.nl/servers/html/index.html based on PDB structure 2HMG.
^{c} Part of receptor binding site.
^{d} Described in [D. C. Wiley, et al., Nature 289 (5796), 373 (1981) and C. A. Smith, et al., J Exp Med 173(4), 953 (1991).

The sites include seven of the eighteen sites identified from earlier data (1983-1997: 121, 138, 142, 145, 186, 226 and 275), which implies their continuous importance in antigenic drift. Some of the other previously found sites still feature an excess of nonsynonymous to synonymous mutations, but the total number of changes is insufficient for significance. The newly identified sites were utilized more strongly in antigenic drift recently than in the previous time period. Thus, there are commonalties, but also some variation in the sites most relevant for antigenic drift during different periods of time. By including more data, the picture of the evolutionary space for antigenic drift can become even more complete.

Repeatedly occurring amino acid mutations associated with antigenic drift and predominant strains of the following season: The phylogeny represents a succession of evolutionary accessible viral variants in the evolution of novel antigenic types. Experimental studies on enzymes can map intermediates in the evolution of a suboptimal allele towards an optimal version on phenotypic fitness landscapes [F. J. Poelwijk, et al., Nature 445 (7126), 383 (2007)]. It has been found that the local landscapes are single peaked (i.e. there is one optimum). There are many unlikely paths on which intermediates show a decrease or no change in fitness and a few favorable ones, which are accessible to Darwinian selection due to successive fitness increases with each introduced mutation. If there is a fitness increase associated with a particular mutation, it occurs more frequently than expected among isolates in that part of the tree. That is, favorable mutations, or mutations that imply a fitness increase appear more often in the phylogenetic tree, while mutations that decrease the fitness occur less often.

The exemplary phylogenetic tree of 1813 isolate sequences of the HA1 subunit of the influenza hemagglutinin contains 1915 mutation events, corresponding to 926 distinct amino acid mutations. 195 amino acid mutations appear multiple times in different lineages of the tree, at 125 sites of the HA1 sequence. These sites are enriched in epitope sites and sites under positive selection (p-value=3.34⁻¹⁰ and 5.32⁻⁷ by hypergeometric distribution), stressing their importance in antigenic drift.

For the amino acid mutations occurring multiple times on edges in the phylogeny, it is important to identify their localization pattern in the phylogeny (i.e. in the tree) and to determine whether they cluster in the tree. This analysis of the clustering behaviour of repeated mutations is performed as follows: The null model is that all mutations in the phylogeny are equally beneficial and there is no significant difference in the path length between the nodes with the same mutation on the incoming edge in the tree in comparison to nodes with other mutations on the incoming edges. It is to determine whether this assumption can explain the observed average path length between nodes with identical mutations. For significance estimation the mutation environment for a set of nodes with the same mutation on the incoming edges of size n is defined as follows: The mutation environment is the distribution over the average path lengths between one of these nodes and n−1 nodes with different mutations on the incoming edges. For each mutation occurring multiple times, the mutation environment distribution was calculated by randomly sampling sets of size n containing one of the n nodes and n−1 nodes with different mutations on the incoming edges from the phylogeny, and computing the average path lengths between all pairs of nodes in the set. Sampling was done 10,000 times. A mutation environment was calculated for each mutation occurring on multiple edges individually as the environment may differ in different parts of the tree, due to the sampling imbalance of the data set in different seasons and geographic locations. From each mutation environment distribution a p-value was obtained for the corresponding set of nodes with the respective mutation, reflecting the significance of their clustering within the phylogeny. The p-value obtained for every multiple occurring mutation from the corresponding mutation environment distribution was corrected for multiple testing with the false discovery rate. Of the 195 mutations occurring multiple times, 140 the corresponding nodes are clustered with the average path length between all pairs of nodes being shorter than the mean of the corresponding mutation environment distribution.

The clustered mutations are enriched in antigenic signature mutations; 27 of the 50 signature mutations are represented (Table 4). In contrast, only one signature change is among the non-clustered repeated mutations, respectively.

TABLE 4
Isolates featuring repeated amino acid mutations with duster in the
phylogeny and match signature mutations of antigenic types. For
each isolate, the amino acid mutation, corresponding antigenic
signature, number of independent occurrences in the phylogeny,
antigenic clade and sampling date of isolate is given.

| | Signature | | | |
|---|---|---|---|---|
| Mutation | clade | Counts | Isolate clade | Sampling date |
| (a) | | | | |
| D124N | JO | 2 | JO | 18/07/1993 |
| D124N | JO | 2 | NA | 17/01/1994 |
| S219Y | JO | 6 | JO | 18/07/1993 |
| S219Y | JO | 6 | FU | 25/12/2002 |
| S219Y | JO | 6 | PA | 6/2/2003 |
| S219Y | JO | 6 | FU | 20/06/2003 |
| S219Y | JO | 6 | FU | 16/08/2003 |
| S219Y | JO | 6 | CA | 16/05/2004 |
| N145K | WU | 4 | NA | 19/04/1993 |
| N145K | WU | 4 | WU | 6/12/1994 |
| N145K | WU | 4 | JO | 25/03/1995 |
| N145K | WU | 4 | TH | 3/1/1996 |
| N262S | WU | 3 | NA | 26/03/1993 |
| N262S | WU | 3 | NA | 14/01/1994 |
| N262S | WU | 3 | WU | 6/12/1994 |
| G275D | BT | 11 | TH | 30/07/1994 |
| G275D | BT | 11 | TH | 10/1/1995 |
| G275D | BT | 11 | WU | 16/07/1996 |
| G275D | BT | 11 | SA | 14/08/1996 |
| G275D | BT | 11 | SA | 13/09/1996 |
| G275D | BT | 11 | PA | 18/10/2001 |
| G275D | BT | 11 | FU | 4/3/2003 |
| G275D | BT | 11 | CA | 27/10/2004 |
| G275D | BT | 11 | CA | 27/06/2005 |
| G275D | BT | 11 | WI | 19/10/2006 |
| G275D | BT | 11 | CA | 5/3/2007 |
| R57Q | BT | 3 | WU | 31/08/1996 |
| R57Q | BT | 3 | SA | 15/11/1996 |
| R57Q | BT | 3 | SY | 19/03/1998 |
| D133N | SA | 2 | SA | 21/01/1996 |
| D133N | SA | 2 | WU | 6/1/1997 |
| G124S | SA | 2 | TH | 7/2/1995 |
| G124S | SA | 2 | SA | 21/01/1996 |
| E158K | SY | 2 | NA | 3/11/1994 |
| E158K | SY | 2 | SY | 8/1/1997 |
| K156Q | SY | 3 | NA | 3/11/1994 |
| K156Q | SY | 3 | JO | 21/04/1995 |
| K156Q | SY | 3 | SY | 8/1/1997 |
| V196A | SY | 3 | NA | 29/07/1994 |
| V196A | SY | 3 | NA | 3/11/1994 |
| V196A | SY | 3 | SY | 8/1/1997 |
| N144I | ST | 2 | ST | 9/6/1999 |
| N144I | ST | 2 | ST | 12/2/2000 |
| Q156H | FU | 3 | FU | 25/04/2001 |
| Q156H | FU | 3 | FU | 7/6/2002 |
| Q156H | FU | 3 | FU | 29/11/2002 |
| S189N | WE | 5 | PA | 11/9/2000 |
| S189N | WE | 5 | PA | 31/12/2001 |
| S189N | WE | 5 | FU | 28/05/2003 |
| S189N | WE | 5 | FU | 7/7/2003 |
| S189N | WE | 5 | FU | 24/07/2003 |
| Y159F | WE | 3 | FU | 22/04/2003 |
| Y159F | WE | 3 | WE | 13/10/2003 |
| Y159F | WE | 3 | FU | 27/11/2003 |
| V226I | CA | 16 | SA | 18/12/1996 |
| V226I | CA | 16 | SA | 18/12/1996 |
| V226I | CA | 16 | SY | 29/06/1997 |
| V226I | CA | 16 | ST | 21/09/2000 |
| V226I | CA | 16 | PA | 30/06/2001 |
| V226I | CA | 16 | PA | 2/5/2002 |
| V226I | CA | 16 | FU | 18/10/2002 |
| V226I | CA | 16 | FU | 25/12/2002 |
| V226I | CA | 16 | NA | 18/03/2003 |
| V226I | CA | 16 | FU | 24/06/2003 |
| V226I | CA | 16 | FU | 10/8/2003 |
| V226I | CA | 16 | FU | 23/12/2003 |
| V226I | CA | 16 | CA | 6/1/2004 |
| V226I | CA | 16 | CA | 8/1/2004 |
| V226I | CA | 16 | FU | 20/01/2004 |
| V226I | CA | 16 | CA | 6/9/2004 |
| K173E | BW | 6 | SY | 29/12/1997 |
| K173E | BW | 6 | ST | 24/12/1999 |
| K173E | BW | 6 | CA | 8/2/2004 |
| K173E | BW | 6 | FU | 22/04/2004 |
| K173E | BW | 6 | WI | 13/02/2006 |
| K173E | BW | 6 | BR | 21/01/2007 |
| V112I | BW | 5 | WU | 1/4/1996 |
| V112I | BW | 5 | NA | 26/11/2002 |
| V112I | BW | 5 | PA | 6/3/2003 |
| V112I | BW | 5 | FU | 22/04/2003 |
| V112I | BW | 5 | CA | 12/3/2004 |
| G50E | BR | 8 | FU | 7/8/2003 |
| G50E | BR | 8 | FU | 16/08/2003 |
| G50E | BR | 8 | FU | 22/09/2003 |
| G50E | BR | 8 | FU | 29/09/2003 |
| G50E | BR | 8 | FU | 29/12/2003 |
| G50E | BR | 8 | WE | 5/8/2004 |
| G50E | BR | 8 | WI | 17/04/2005 |
| G50E | BR | 8 | BR | 23/10/2006 |
| D124N | JO | 2 | JO | 18/07/1993 |
| D124N | JO | 2 | NA | 17/01/1994 |
| S219Y | JO | 6 | JO | 18/07/1993 |
| S219Y | JO | 6 | FU | 25/12/2002 |
| S219Y | JO | 6 | PA | 6/2/2003 |
| S219Y | JO | 6 | FU | 20/06/2003 |
| S219Y | JO | 6 | FU | 16/08/2003 |
| S219Y | JO | 6 | CA | 16/05/2004 |
| N145K | WU | 4 | NA | 19/04/1993 |
| N145K | WU | 4 | WU | 6/12/1994 |
| N145K | WU | 4 | JO | 25/03/1995 |
| N145K | WU | 4 | TH | 3/1/1996 |
| N262S | WU | 3 | NA | 26/03/1993 |
| N262S | WU | 3 | NA | 14/01/1994 |
| N262S | WU | 3 | WU | 6/12/1994 |
| G275D | BT | 11 | TH | 30/07/1994 |
| G275D | BT | 11 | TH | 10/1/1995 |
| G275D | BT | 11 | WU | 16/07/1996 |
| G275D | BT | 11 | SA | 14/08/1996 |
| G275D | BT | 11 | SA | 13/09/1996 |
| G275D | BT | 11 | PA | 18/10/2001 |
| G275D | BT | 11 | FU | 4/3/2003 |
| G275D | BT | 11 | CA | 27/10/2004 |
| G275D | BT | 11 | CA | 27/06/2005 |
| G275D | BT | 11 | WI | 19/10/2006 |
| G275D | BT | 11 | CA | 5/3/2007 |
| R57Q | BT | 3 | WU | 31/08/1996 |
| R57Q | BT | 3 | SA | 15/11/1996 |
| R57Q | BT | 3 | SY | 19/03/1998 |
| D133N | SA | 2 | SA | 21/01/1996 |
| D133N | SA | 2 | WU | 6/1/1997 |
| G124S | SA | 2 | TH | 7/2/1995 |
| G124S | SA | 2 | SA | 21/01/1996 |
| E158K | SY | 2 | NA | 3/11/1994 |
| E158K | SY | 2 | SY | 8/1/1997 |
| K156Q | SY | 3 | NA | 3/11/1994 |
| K156Q | SY | 3 | JO | 21/04/1995 |
| K156Q | SY | 3 | SY | 8/1/1997 |
| V196A | SY | 3 | NA | 29/07/1994 |
| V196A | SY | 3 | NA | 3/11/1994 |
| V196A | SY | 3 | SY | 8/1/1997 |
| N144I | ST | 2 | ST | 9/6/1999 |
| N144I | ST | 2 | ST | 12/2/2000 |
| Q156H | FU | 3 | FU | 25/04/2001 |
| Q156H | FU | 3 | FU | 7/6/2002 |
| Q156H | FU | 3 | FU | 29/11/2002 |
| S189N | WE | 5 | PA | 11/9/2000 |
| S189N | WE | 5 | PA | 31/12/2001 |
| S189N | WE | 5 | FU | 28/05/2003 |
| S189N | WE | 5 | FU | 7/7/2003 |
| S189N | WE | 5 | FU | 24/07/2003 |
| Y159F | WE | 3 | FU | 22/04/2003 |
| Y159F | WE | 3 | WE | 13/10/2003 |
| Y159F | WE | 3 | FU | 27/11/2003 |
| V226I | CA | 16 | SA | 18/12/1996 |
| V226I | CA | 16 | SA | 18/12/1996 |
| V226I | CA | 16 | SY | 29/06/1997 |
| V226I | CA | 16 | ST | 21/09/2000 |
| V226I | CA | 16 | PA | 30/06/2001 |
| V226I | CA | 16 | PA | 2/5/2002 |
| V226I | CA | 16 | FU | 18/10/2002 |
| V226I | CA | 16 | FU | 25/12/2002 |
| V226I | CA | 16 | NA | 18/03/2003 |
| V226I | CA | 16 | FU | 24/06/2003 |
| V226I | CA | 16 | FU | 10/8/2003 |
| V226I | CA | 16 | FU | 23/12/2003 |
| V226I | CA | 16 | CA | 6/1/2004 |
| V226I | CA | 16 | CA | 8/1/2004 |
| V226I | CA | 16 | FU | 20/01/2004 |
| V226I | CA | 16 | CA | 6/9/2004 |
| K173E | BW | 6 | SY | 29/12/1997 |
| K173E | BW | 6 | ST | 24/12/1999 |
| K173E | BW | 6 | CA | 8/2/2004 |
| K173E | BW | 6 | FU | 22/04/2004 |
| K173E | BW | 6 | WI | 13/02/2006 |
| K173E | BW | 6 | BR | 21/01/2007 |
| V112I | BW | 5 | WU | 1/4/1996 |
| V112I | BW | 5 | NA | 26/11/2002 |
| V112I | BW | 5 | PA | 6/3/2003 |
| V112I | BW | 5 | FU | 22/04/2003 |
| V112I | BW | 5 | CA | 12/3/2004 |
| G50E | BR | 8 | FU | 7/8/2003 |
| G50E | BR | 8 | FU | 16/08/2003 |
| G50E | BR | 8 | FU | 22/09/2003 |
| G50E | BR | 8 | FU | 29/09/2003 |
| G50E | BR | 8 | FU | 29/12/2003 |
| G50E | BR | 8 | WE | 5/8/2004 |
| G50E | BR | 8 | WI | 17/04/2005 |
| G50E | BR | 8 | BR | 23/10/2006 |

Many of the clustered amino acid mutations emerge in independent lineages one to three years before establishment of a novel antigenic type. An example of such a mutation is G50E of the Brisbane clade signature. Prior to the establishment of Brisbane, G50E already occurred in other lineages of the Wisconsin clade in 2006, the clade Wellington in 2004 and Fujian in 2003. In the Wisconsin clade, it is accompanied by the K140I mutation, giving rise to clade Brisbane. The California and Wellington signature mutation S189N appeared independently three times in the precursor Fujian in 2003 and twice in the Panama clade, in 2000 and 2001. For a certain window in time, there is a distinct advantage associated with these mutations on the viral fitness landscape; already before they occur in combination with others in a novel and dominating antigenic type.

Geographic spread and origin of successful strains: Seasonal influenza infections predominantly occur in the winter season of the northern and southern hemispheres, but year-round in the tropics. Understanding the epidemiology of the virus requires resolving the details of viral geographic spread. As sampling dates and geographic location are available, visualization of these properties in combination with the antigenic clade structure conveniently reveals information on the timing and geographic prevalence of the successively emerging viral lineages (FIG. 4).

Implications: For influenza A, the phylogeny identifies (i) repeated amino acid mutations significantly associated with antigenic drift which allow the recommendation of suitable vaccine strains matching the predominant viral phenotype of the upcoming influenza season (ii) the sites most relevant for antigenic drift and (iii) clades corresponding to the dominating antigenic types of the virus during the analyzed time span.

It is therefore apparent that the phylogeny of the present invention is a powerful tool for resolving the evolutionary history of the influenza virus. Thus, a further aspect of the invention relates to a method for the production of a vaccine against a fast-evolving entity by analyzing biological sequences of a plurality of isolates to generate a phylogenetic tree, the recommendation or selection of a suitable individual isolate of the entity as a candidate for the forthcoming virus outbreak season, and the preparation of a vaccine based on such a recommendation or selection.

The recommendation or selection of an individual isolate of the fast-evolving entity comprises the steps of (i) computation of a phylogenetic tree from sequences available up to the point in time where the decision is to be made, (ii) identifying repeated mutations in a phylogenetic tree, (iii) identifying the number of repeated mutations occurring in each isolate and (iv) the selection of an isolate as a vaccine candidate when the isolate bears the maximum number of repeated mutations that only occur on the incoming edges of nodes with timestamps from a time-span of 3 or 4 years before the season in which the vaccine is to be produced and are located within an antigenic site of the hemagglutinin. Specifically, an isolate is selected as a vaccine in step (iv) when (a) the isolate sequence bears the maximum number of repeated amino acid mutations that occurred within a time-span of 4 years before the season in which the vaccine is to be produced, or (b) the isolate sequence bears the maximum number of repeated amino acid mutations that are located in antigenic sites and occurred within a time-span of 4 years before the season in which the vaccine is to be produced, or (c) the isolate sequence bears the maximum number of repeated amino acid mutations that cluster in the phylogeny, are located in antigenic sites and occurred within a time-span of 3 years before the season in which the vaccine is to be produced, or (d) the isolate sequence bears the maximum number of repeated amino acid mutations that cluster in the phylogeny, are located in sites determined experimentally to change the viral phenotype and occurred within a time-span of 4 years before the season in which the vaccine is to be produced.

In another embodiment of this aspect of the invention, the clustering behaviour of amino acid mutations can be taken into account in the selection process. That is, an isolate is selected if when the isolate bears the maximum number of repeated amino acid mutations which (i) cluster with the average path length between all pairs of nodes being shorter than the mean of the corresponding mutation environment distribution calculated as described above, (ii) occur only on the incoming edges of nodes with time stamps from a time-span of 3 or 4 years up to the season in which the vaccine is to be produced and (iii) are located within an antigenic site of the hemagglutinin.

In another embodiment of this aspect of the invention, the geographic spread can be taken into account in the selection process. That is, if a vaccine for the northern hemisphere is desired and more than one otherwise equally suitable isolates from different geographic regions have been identified, the preference is given to the strain which was sampled from the northern hemisphere.

In a further embodiment, the weight of the nodes can be taken into account in the selection process. The weight of a node is determined by the number of its direct progeny, e.g. a “parent” isolate with 20 direct descendants is more suitable as a vaccine than an isolate with only 3 descendants (provided the remaining parameters outlined above are identical).

In a further embodiment the prediction on the basis of the clustered repeated mutations can be combined with the calculation of Pₑₚᵢₜₒₚₑ. Pₑₚᵢₜₒₚₑ is a variable that measures the antigenic distance between two influenza strains. For a given viral strain, the change in antigenicity relative to a reference strain is related to the genetic change in the antigenic site which exhibits the most change of the viral hemagglutinin surface protein (the dominant antigenic site). Pₑₚᵢₜₒₚₑ is the normalized difference between the vaccine strain used in a particular season and the analyzed strain within the dominant antigenic site. It was shown that Pₑₚᵢₜₒₚₑ has a positive correlation with vaccine efficacy [Gupta, V. et al., Vaccine 24(18), 2006:3881-8]. The results of an exemplary analysis of biological isolate sequences are shown in Tables 5 and 6.

TABLE 5
Repeated mutations used to select isolate sequences as
vaccine strain recommendations for seasons in which a
vaccine strain update was required.

| Year and | |
|---|---|
| hemisphere | Mutations |
| 95/96N | N246S, (A138S, N145K, N262S) |
| 98/99N | K207Q, L164Q, R220G, R229I, (R142G, T192I, V223I) |
| 1999S | D271N, R229K, S186G, (K92T, T192I) |
| 2002S | D291G, I288V, I58V, K2E, R220X, R229K, R269K, |
| | T167I, (T10M) |
| 02/03N | E62G, G225D, I267V, N144T, Q156H, W222R, (A272T) |
| 03/04N | G50E, G78S, I34L, K145N, K92R, N144D, |
| | S189N, S193N, S45I, Y159F, |
| | (D188E, D77E, L3F, N312K, N8K, V226X) |
| 2004S | D188N, G186V, G50E, I230M, K145N, |
| | K92R, S193N, S199P, S262N, Y105H, |
| | Y159F, (A106V, A304P, G186S, T12M) |
| 2005S | G186V, G50E, I217V, I67M, N145S, N216S, |
| | P227S, S124N, T131N, (N165K, T10R) |
| 05/06N | D225N, G50E, K276N, K27R, K83R, |
| | K92R, L66I, M168L, N216S, N225D, |
| | Q327K, S124N, S199A, T128A, Y195H |
| 2006S | D188G, D225N, E50G, N225D |
| 06/07N | D188Y, E50G, G186V, G50E, I34V, |
| | K140I, K259R, K326R, L194P, N225D, |
| | N96K, P221X, R261Q, S124N, Y105H, Y195X |

*Epitope Sites in bold font,
N = northern hemisphere,
S = southern hemisphere
Column 2 list all repeatedly occurring mutations identified from phylogenies constructed from the available HA1 sequences up to the season in which the vaccine strain recommendation is to be made (e.g. with data up to March of 1995 for the 95/96N prediction), which occur only on incoming edges of nodes with a time stamp from within the last four years.
In brackets the non-clustered repeated mutations are given.
Mutations localizing to the antigenic sites of the HA1 protein are shown in bold font.
Mutations or sequence changes given in the table have the format “previous_aminoacid” “sequence_position” “new_aminoacid”, e.g. G186V signifies that glycine at position 186 is replaced by valine. Sequences in the test set are scored with the mutation list for the respective season, adding one count for every match of the ‘new_aminoacid’ at “sequence position” (e.g. for D188Y, the score for a sequence is incremented by one if the tested sequence features a “Y” at position 188).

TABLE 6
Accuracy of vaccine strain recommendations for seasons in which a vaccine strain update was required.

| | | | | | | crMs | | crMs | | rMs | | rMs | |
|---|---|---|---|---|---|---|---|---|---|---|---|---|---|
| Season | From | To | Test | Predominant | WHO | All | | Epitope | | All | | Epitope | |
| 96/97N | October 1996 | March 1997 | 11 | WU | WU | 1 | (2) | 1 | (2) | 1 | (2) | 1 | (2) |
| 99/00N | October 1999 | March 2000 | 47 | PA | SY | 0.875 | (8) | 1 | (4) | 1 | (9) | 1 | (5) |
| 03/04N | October 2003 | March 2004 | 20 | FU | PA | 1 | (1) | 1 | (1) | 1 | (1) | 1 | (1) |
| 04/05N | October 2004 | March 2005 | 70 | CA | FU | 0.5 | (2) | 0.5 | (2) | 0.5 | (2) | 0.5 | (2) |
| 06/07N | October 2006 | March 2007 | 68 | WI | WI | 1 | (2) | 1 | (1) | 1 | (2) | 1 | (1) |
| 07/08N | October 2007 | March 2008 | 77 | BR | WI | 1 | (1) | 1 | (8) | 1 | (1) | 1 | (8) |
| 2000S | April 2000 | September 2000 | 5 | PA | PA | 0 | (1) | 0.5 | (2) | 0 | (1) | 0 | (1) |
| 2003S | April 2003 | September 2003 | 46 | FU | PA | 0 | (1) | 0 | (4) | 0 | (1) | 0 | (4) |
| 2005S | April 2005 | September 2005 | 44 | CA | WE | 1 | (1) | 1 | (1) | 1 | (1) | 1 | (1) |
| 2006S | April 2006 | September 2006 | 47 | WI | CA | 0 | (1) | 0 | (1) | 0 | (1) | 0 | (1) |
| 2007S | April 2007 | September 2007 | 4 | BR | WI | 0 | (2) | 0 | (2) | 0 | (2) | 0 | (2) |

| | | | | | |
|---|---|---|---|---|---|
| Accuracy | 0.27 | 0.58 | 0.64 | 0.59 | 0.59 |

A strain is selected one year before. Column 1 gives the seasons for which a vaccine update was required for the northern hemisphere (N) and southern hemisphere (S), respectively. The number of sequenced isolates from the corresponding season of the preceding year which were available for testing with our method is given in column 4. The predominating strain in the season for which the vaccine was to be produced is shown in column 5, WHO influenza vaccine strain recommendation for the season in column 6, wherein boldface signifies a correct recommendation and italics signifies a wrong recommendation. Columns 7-10 show for the sequences recommended with our method the percentage matching the phenotype-signature mutations of the dominating strain for the season, wherein boldface signifies a correct recommendation and italics signifies a wrong recommendation. In brackets the number of recommended sequences is given. Sequences were scored as described above, based on the number of repeated mutations (rMs, columns 9, 10) or clustered repeated mutations (crMs, columns 7, 8) which occurred in the four preceding years in a phylogeny built from all HA1 sequences available until the end of the season of the preceding year (e.g. from sequences available until the end of March of 1995 for the 95/96N vaccine strain recommendation). Columns 7 and 9 show the results under consideration of mutations at all HA1 positions, columns 8 and 10 show the results under consideration of mutations within the antigenic sites of the hemagglutinin. Antigenic sites correspond to those defined in Wilson and Cox (1990), Structural Basis of Immune Recognition of Influenza Virus Hemagglutinin, Annual Review of Immunology, VOL 8. pages 737-771.

In a further embodiment, the fast-evolving entity selected by the inventive methods can be used for the preparation of a vaccine.

A further embodiment of the present invention relates to a computer implemented method of pattern-based stochastic search for maximum parsimony phylogenies (pTree). This embodiment reconstructs a phylogenetic tree from a set of biological sequences such as protein sequences or nucleic acid sequences, preferably DNA sequences.

The input of the algorithm of this embodiment is an initial set of DNA sequences whereas each sequence consists of characters A, T, G, C, -. These initial sequences are also referred to as “original sequences” or “originals”. These original sequences can represent isolates from a fast evolving entity such as influenza A virus. These originals can also represent artificial sequences that are to be analyzed by the algorithm. Sequences that are generated by the algorithm itself (i.e. the sequences that are inferred by the algorithm) are denoted as “intermediate sequences”, “inferred sequences” or “intermediates”. Accordingly the term “original node” refers to a node of a phylogenetic tree that is associated with an original input sequence, while an “inferred node” relates to a node of a phylogenetic tree that is associated with an inferred (artificial) sequence.

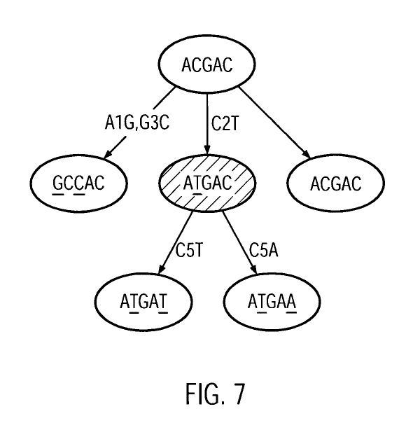

The output of the algorithm is an unrooted, multifurcating phylogenetic tree. Associated with each node is a DNA sequence (original or inferred) and each edge (or branch) is labeled with a list of substitutions, so that they represent the differences in DNA between the two nodes incident to the edge. The cost (or length) of the tree is the sum of the number of substitutions on all of the edges. Each leaf in the tree is associated with one original sequence, each internal node is associated with an intermediate sequence. Internal nodes may be associated with intermediates that are identical to an original, in which case one of its neighbors is a leaf associated with that original sequence. FIG. 7 shows an example of an output tree, with original nodes in white and inferred intermediate nodes shaded.

In this method a first step is to compute a matrix of pairwise evolutionary distances (DM) for the input sequences under a suitable evolutionary model, such as e.g. the Jukes-Cantor model. The resulting distance matrix is the input to a distance-based tree reconstruction method such as the Neighbor-joining (NJ) algorithm that outputs a tree. The corresponding tree metric (TM) is the input to a minimum spanning tree (MST) algorithm. The resulting MST is rooted by choice of an arbitrary node as the root node and then new intermediate nodes are inferred with associated sequences by searching for repeated substitution patterns on edges incident to the same internal node. These intermediates are included in the inference of a phylogenetic tree based on an MST algorithm in an iterative fashion until no more new intermediates are found. The root node can be a leaf node or an internal node.

A “leaf node” is any node that is only connected to one other node in the tree. By selecting a root node, the tree is at the same time oriented. “Oriented” means that by choice of a root node, the tree is given a direction (represented by directed arrows in the Figures). Starting from the root (which id defined as having no incoming edges), now, every internal node has an incoming edge, and one or more outgoing edges, to the leaf nodes, which have an incoming edge, but no outgoing edges. i.e. every edge (branch) in the tree can now be identified as being an incoming edge or an outgoing edge for each node in the tree. A root node has only outgoing edges. Hence, “root & orient” in the following pseudocode samples reflect the arbitrary choice of a leaf node as root node and the resulting orientation of the tree that comes along with that selection.

The following lines summarize the method in pseudocode.

| | |
|---|---|
| 1. | DM ← compute distance matrix from original DNA sequences |
| 2. | NJ tree ← compute NJ tree from DM |
| 3. | TM ← compute tree metric (from NJ tree) |
| 4. | MST ← compute MST & root & orient |
| 5. | loop: infer new intermediate sequences and reconstruct the tree |
| 6. | output the phylogenetic tree |

Next, the step of inferring intermediates & repeated substitution patterns is described.

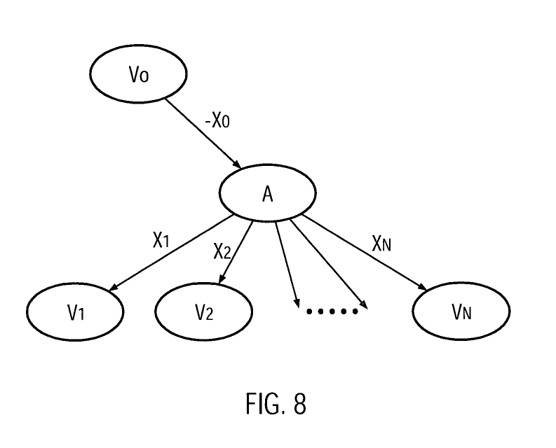

The step of inferring intermediates & repeated substitution patterns is exemplified with FIG. 8. Node V₀ is a parent of an internal node A and nodes V₁, V₂, . . . , V_{N} are children of A. Each Xᵢ is a set of substitutions whereas a substitution is defined as a triple (char from, position, char to) that denotes a position which differs between sequences A and Vᵢ as seen in direction from A to neighboring node Vᵢ (e.g. C2T would signify that C at position 2 of a sequence in a parent node is substituted with T in the child node). These triples are also named “edge substitution sets”. The negative sign before X₀ denotes the fact that −X₀ is a set of point mutations as seen from V₀ to A whereas X₀ is a set of point mutations as seen from A to V₀ (hence, the above C2T would have to be written as T2C).

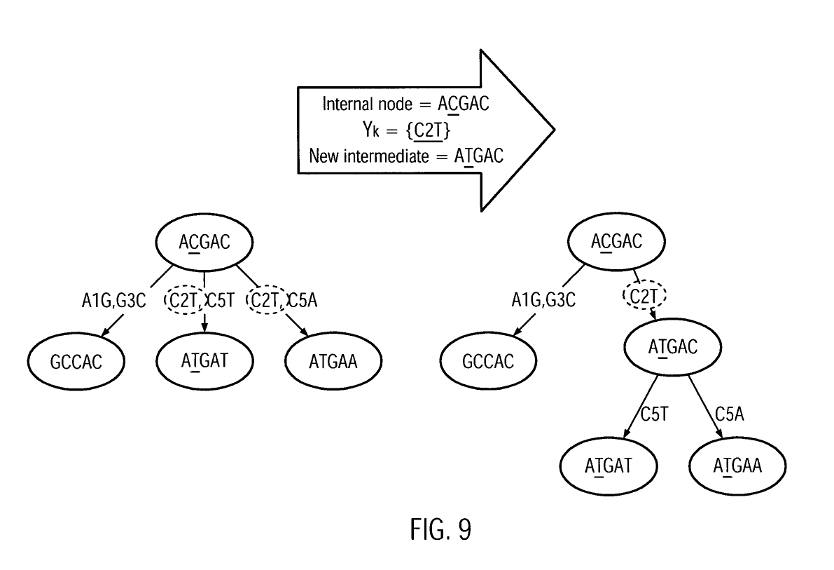

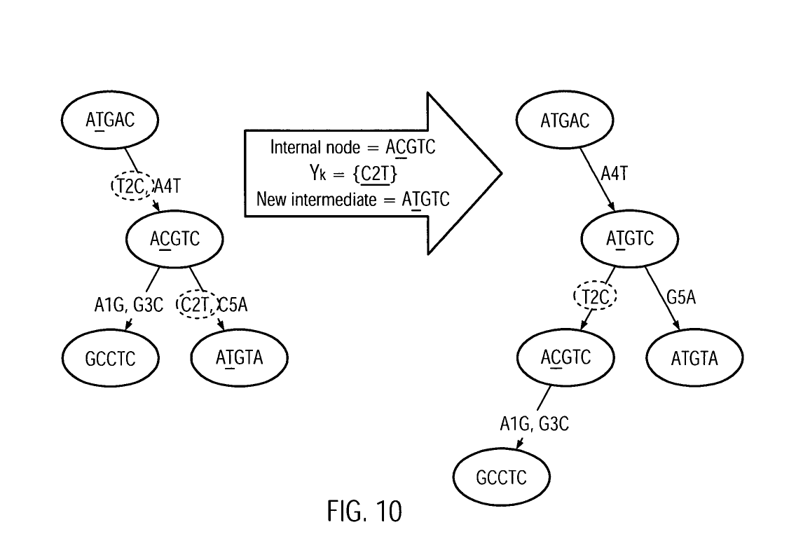

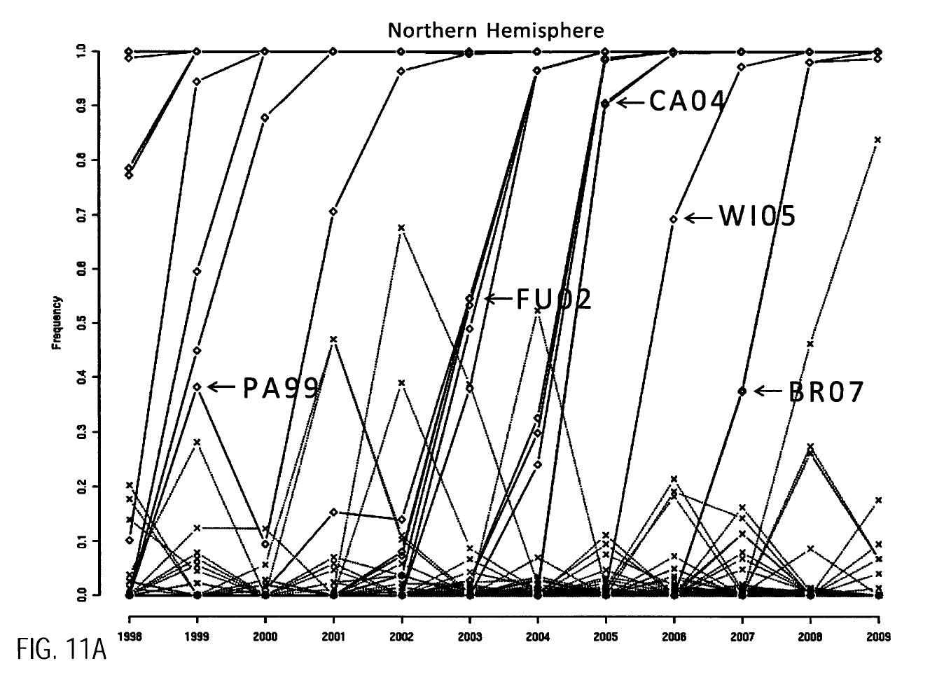

If there exists i, jε[0 . . . N] so that i≠j & Xᵢ∩Xⱼ=Yₖ & Yₖ≠Ø then a new intermediate node Iₖ for all distinct sets Yₖ is inferred. In other words, if there exists a pair of edges from an internal node A to any pair (Vᵢ, Vⱼ) of two neighboring nodes of A for which the intersection Yₖ of the corresponding edge substitution sets Xᵢ and Xⱼ is not empty, then we infer a new intermediate node Iₖ, (hatched nodes in FIGS. 9 and 10) for all distinct sets Yₖ. This condition is called “repeated substitution pattern”. The sequence associated with the new intermediate node Iₖ is derived from the sequence of node A by applying the substitutions from Yₖ.

Two examples of resolving repeated substitution patterns into intermediates are given in FIGS. 9 and 10. They show how the repeated substitution pattern can be found, how the new intermediate node is inferred and how the new inferred node is included in the tree topology as well as what sequence the new inferred node is associated with. This process is also referred to as intermediate sampling.

By the aforementioned intermediate sampling, theoretically, a lot of intermediate nodes can be inferred. This may substantially slow down the overall performance of the algorithm since one internal node that has n neighbors can theoretically produce up to n*(n−1)/2 intermediate nodes. Therefore, in a preferred embodiment of this aspect of the invention the number of inferred intermediates is limited by one of the following strategies:

The first strategy is to randomly choose a limited number of intermediates, wherein this number is for example set equal to the number of original input sequences. The non-elected inferred intermediates are removed from the data set.

The second strategy is to define a ranking function that assigns a number (or value) to each inferred node meaning how good a particular intermediate is. Then a limited number of intermediates is chosen according to the ranking function. The ranking function can be, e.g., equal to the size of the corresponding mutation set (set size refers to the number of substitutions in the set) or equal to the cost reduction estimate for insertion of the intermediate into the local tree topology based on the substitution sets for all edges incident to the internal node A defined above.

The third strategy first optionally reduces the number of intermediates by applying the first or second strategy and then combines this remaining list of intermediates with the neighbors of the internal node and the corresponding internal node. Then one can determine the best choice of intermediates considering the local tree topology using the algorithm that is defined in lines 4-9. of the pseudocode in the following section (Inferring intermediates by depth-first-search DFS).

Definition of node status and types: To facilitate the algorithm description one defines for each node its status and type. As described above, one distinguishes between two types of nodes: original nodes that were given as an input and inferred nodes that were inferred during the run of the algorithm. Specifically, one assigns to each node a status that can be 0, 1 or 2. An original node has status 1. An intermediate node has status 2 if it is identical to a sequence with status 1 or 2. An intermediate node that represents a new sequence has status 0.

Inferring intermediates by depth-first-search (DFS): Now that one knows how to find the repeated substitution pattern and how to infer new intermediates at some internal node of a tree, the completion of the inference loop is described. The tree is rooted at an arbitrary leaf node. Then a depth-first search (DFS) algorithm is run from the root to search for repeated substitution patterns at each internal node and if a repeated substitution pattern is found, a new intermediate node is inferred. The status for each new inferred intermediate is set and then all newly inferred intermediates with status 2 are removed. Then the distance matrix (DM) and the minimum spanning tree (MST) is recomputed with the resulting data set (i.e. considering all original and inferred vertices). After that all inferred nodes with status=0, in-degree=1 and out-degree=0 or 1 are deleted. The expression “in-degree=1” means that the node has one incoming edge, the expression “out-degree=0 or 1” means that the node has either no or one outgoing edges. Then we try again to find new intermediate vertices in the next loop.

The following lines summarize the inferring process in pseudocode:

Inferring process

| | |
|---|---|
| 1. | loop: |

| | |
|---|---|
| 2. | Intermediates ← run DFS to get new intermediates |
| 3. | if no new intermediates are found (Intermediates == Ø) then |
| | break the loop (1) |
| 4. | set the status of inferred intermediates |
| 5. | remove intermediates that represent the same DNA sequence as |
| | some node we already had |
| 6. | loop: |

| | |
|---|---|
| 7. | recomputed DM & MST (& root & orient) |
| 8. | remove intermediates with status = 0 & in-degree = |
| | 1 & out-degree = 0 |
| 9. | remove intermediates with status = 0 & in-degree = |
| | 1 & out-degree = 1 |
| 10. | if (no intermediate was removed at (9)) then break the loop |
| | 6. |

### Basic Algorithm Overview

Now the basic algorithm of this embodiment will be described in pseudocode. The algorithm description is divided into three phases: initializing step, update step and final step.

Initializing step

| | |
|---|---|
| 1. | remove duplicate original vertices (in terms of the same DNA), |
| | first occurrences remain |
| 2. | compute distance matrix (DM) |
| 3. | compute NJ tree |
| 4. | set NJ matrix according to distances in the NJ tree |
| 5. | compute MST, root & orient the tree |

Update step

| | |
|---|---|
| 1. | loop: |

| | |
|---|---|
| 2. | infer new intermediates |
| 3. | if (no intermediate was inferred) then break the loop (1) |
| 4. | set status of inferred vertices |
| 5. | remove duplicate vertices (new duplicate intermediates are |
| | removed) |
| 6. | loop: |

| | |
|---|---|
| 7. | recompute the DM & MST (& root & orient) |
| 8. | remove intermediates with status=0, in-degree=1 and out- |
| | degree=0 |
| 9. | remove intermediates with status=0, in-degree=1 and out- |
| | degree=1 |
| 10. | if no intermediate was removed at step (9) then break the |
| | loop (6) |

Final step

| | |
|---|---|
| 1. | add duplicate original vertices that were removed at the |
| | Initializing step |
| 2. | remove intermediates status= 0 or 2, in-degree=1 and out-degree=0 |
| 3. | move originals to leaves (*) |
| 4. | output the tree |

(*) Note:
For each original node that is an internal node create its copy and make an arrow from the original node to its copy. This step ensures that all original nodes will be leaves after this operation completes.

As a further embodiment of the method an extended algorithm with masking will now be described. In a preferred embodiment of the method, the above method (basic algorithm) is modified by introducing a modification that iterates modified basic algorithm several times with different “masking”, maintaining the best solution that has been found so far.

“Masking” is a set of sites/positions in DNA sequences. The idea of masking is that one masks some sites/positions in DNA sequences that will not be considered in the further computation. For instance if the 3^{rd} and 5^{th} site/position is masked (masking={3,5}) in the input set {“AGTCCT”, “TGCAGT”}, a masked set {“AGXCXT”, “TGXAXT”} results. While the distance between the first and second (unmasked) sequences is given by the mutation set {“A1T”, “T3C”, “C4A”, “C5G”}, the distance between their masked counterparts is given by the mutation set {“A1T”, “C4A”}.

Each node represented exactly one sequence so far. From now on each node will represent two sequences: one is the original unmasked sequence and the other is the masked sequence. Furthermore, if a new intermediate is inferred, it will consist of two sequences: one unmasked and one masked.

One embodiment of this aspect of the invention pertains to the computation of a random masking. In the following, the creation of a random masking (a set of sites/positions in DNAs that will be masked) is described. The input of this subroutine is a set of unmasked sequences and the parameter “M” that denotes how many sites/positions will be masked. The subroutine first determines candidate sites/positions that could be masked and then randomly chooses (at most*) M of them. M is a user-set value which as a default is preferably set to 10. M can be any integer between 1 and 100, preferably 5-20, most preferably 10. A skilled person knows to set a workable value for M in view of the length and diversity of the sequences that are under consideration for masking. In the case of assessing influenza A hemagglutinin sequences, the value is preferably set to 10.

The following algorithm describes in more detail how the random masking is performed:

| | |
|---|---|
| 1. | for each position |

| | |
|---|---|
| 2. | a : = t := g := c:= 0 |
| 3. | for each DNA sequence |

| | |
|---|---|
| 4. | if (at the position in a DNA sequence is A, T, G or C) |
| | then a++, t++, g++ or c++ |

| | |
|---|---|
| 5. | if (at least two out of a, t, g, c are >= 2) then this position |
| | is a candidate position |

| | |
|---|---|
| 6. | choose randomly (at most*) M positions (out of all candidate |
| | positions) |

Note:
*It can happen that the number of candidate positions is M* and M* < M, in this case we choose these M* positions.

The extended algorithm overview: A further embodiment relates to an extended algorithm of the method described above. This extended algorithm calls a slightly modified basic algorithm several times in two different versions. The first version computes the tree without masking, i.e., it considers “only” unmasked sequences. The second version computes the tree with some random masking, i.e., it does not consider masked sites/positions when it is searching for repeated substitution patterns. Both versions have in common that they output the tree in terms of unmasked sequences, so that the cost of the different trees can be compared.

The algorithm has two additional input parameters. The first is the number of sites that will be masked and the second is the parameter N of the main for-loop (see line 3 of the pseudocode below):

| | |
|---|---|
| 1. | compute a tree Tree0 (without masking) |
| 2. | Tree_min := Tree0 |
| 3. | for ( i=0; i< N; i++) |

| | |
|---|---|
| 4. | compute a tree Tree_temp (with some random masking) |
| 5. | put together: original input sequences + intermediates (status0) |
| | from Tree_min & Tree_temp |
| 6. | compute Tree_combined (with some random masking) with |
| | input sequences defined at (5) |
| 7. | if (cost(Tree_combined) < cost(Tree_min))) then { Tree_min |
| | := Tree_combined; i :=0; } |

| | |
|---|---|
| 8. | output the Tree_min |

Complete extended algorithm: In the following, the complete algorithm description will be given in pseudocode. The description is divided into two parts: “the main algorithm” and subroutine “compute partial tree”. Note that the type and status of nodes or vertices does not change during the algorithm. Once the status of a node or vertex is determined, it will stay the same till the end of the algorithm. The status of a node or vertex is determined in terms of unmasked sequences.

The main algorithm

| | |
|---|---|
| 1. | remove duplicate vertices (in case of identical DNA |
| | sequences, one is chosen at random to remain in the |
| | working set of vertices). |
| 2. | Tree0 ← call compute partial tree (without masking, |
| | input: originals) |

| | |
|---|---|
| 3. | Tree_min := Tree0 |
| 4. | i := 0; |

| | |
|---|---|
| 5. | while (true) |
| 6. | if (i == N) then |

| | |
|---|---|
| | { break the loop (5)} |
| 7. | Tree_temp ← call compute partial tree (with some |
| | random masking, input: originals) |
| 8. | Tree_combined ← call compute partial tree (with |
| | some random masking, input: originals + |
| | intermediates status=0 from Tree_temp + |
| | intermediates status=0 from Tree_min) |
| 9. | if (cost(Tree_combined) < cost(Tree_min))) then |

| | |
|---|---|
| 10. | { i :=0; Tree_min := Tree_combined; } |

else

| | |
|---|---|
| 11. | {i++; } |
| 12. | recompute DM & MST according to the |
| | unmasked sequences from Tree_min |

| | |
|---|---|
| 13. | add original vertices removed at step (1) to the |
| | Tree_min |

| | |
|---|---|
| 14. | remove intermediate with status = 0 or 2, in-degree=1, out- |
| | degree=0 |
| 15. | move originals to leaves |
| 16. | output the Tree_min |

Compute partial tree (subroutine)

| | |
|---|---|
| 1. | remove duplicate vertices in terms of masked sequences & store |
| | removed original vertices |
| 2. | DM ← compute distance matrix (consider masked sequences) |
| 3. | TM ← compute tree metric from (NJ) tree |
| 4. | MST ← compute MST & root & orient |
| 5. | loop: |

| | |
|---|---|
| 6. | intermediates ← search for repeated substitution pattern |
| | (considering masked sequences) |
| 7. | if (Intermediates == Ø) then break the loop (5) |
| 8. | set the status of the inferred intermediates (considering |
| | unmasked sequences) |
| 9. | remove duplicate intermediate vertices (considering masked |
| | sequences) |
| 10. | loop: |

| | |
|---|---|
| 11. | recomputed DM & MST (considering masked sequences) |
| 12. | remove intermediates status=0, in-degree=1, out-degree=0 |
| | (considering masked s.) |
| 13. | remove intermediates status=0, in-degree=1, out-degree=1 |
| | (considering masked s.) |
| 14. | if (no intermediate removed at (13)) break the loop (10) |

| | |
|---|---|
| 15. | add original vertices removed at step (1) |
| 16. | recompute DM & MST (considering masked s.) |
| 17. | output MST |

A further embodiment of the invention pertains to the identification of future predominant lineages for human influenza A virus from phylogenetic trees generated by the above described pattern-based stochastic search for maximum parsimony phylogeny.

Here it is demonstrated how to identify future predominant variants for human influenza A based on a gene allele predominance plot inferred from a phylogenetic tree and reconstructed sequence intermediates for this tree. In this case the phylogenetic tree has been obtained by the pattern-based stochastic search for maximum parsimony, described above.

The early identification of the next predominant influenza strain is important with regard to the problem of selecting a viral strain for production of the influenza vaccine. This strain should be well matched antigenically to the predominant viral variant in the following influenza season. Due to the time requirements of the production and distribution process, this strain has to be identified almost one year in advance from available genetic, antigenic and phenotypic surveillance data for seasonal influenza A viruses from around the globe.

From 4913 exactly dated sequences of the major viral surface protein (hemagglutinin) for the influenza A (H3N2), a phylogenetic tree was inferred, the sequences of the ancestral nodes were reconstructed and substitution events were inferred for the respective edges (branches). From this reconstruction, one defines and infers a ‘gene allele predominance plot’, which shows the frequencies of gene alleles in the analyzed viral population over time. Every allele is defined by a single substitution event on the branch of this phylogeny. We limit our analysis to alleles defined by substitutions in the five known antibody binding sites of the influenza hemagglutinin in this phylogenetic tree.

While the present analysis has been performed on hemagglutinin, it can also be performed on any allele that has been shown to influence the virulence of influenza A. Furthermore, the present method can also be applied to antigenic alleles of other fast evolving entities, such as viruses, e.g. other influenza strains, preferably Influenzavirus A, B, C, Thogotovirus and Isavirus (all Ortho-myxoviridae), and more preferably on subtypes of influenza virus A such as H1N1, H1N2, H2N2, H3N1, H3N2, H3N8, H5N1, H5N2, H5N3, H5N8, H5N9, H7N1, H7N2, H7N3, H7N4, H7N7, H9N2 or H10N7.

The allele frequency in one year is defined as the relative number of isolates (i.e. divided by the number of all isolates that were sampled in the given year) in the subtree of the branch based on which the allele is defined. Years of the Northern hemisphere are defined to start at the 1st of April and end at the 31st of March, at the end of the winter season. For the Southern hemisphere, the year is defined to start on the 1st of October and end at the 30th of September in the following year.

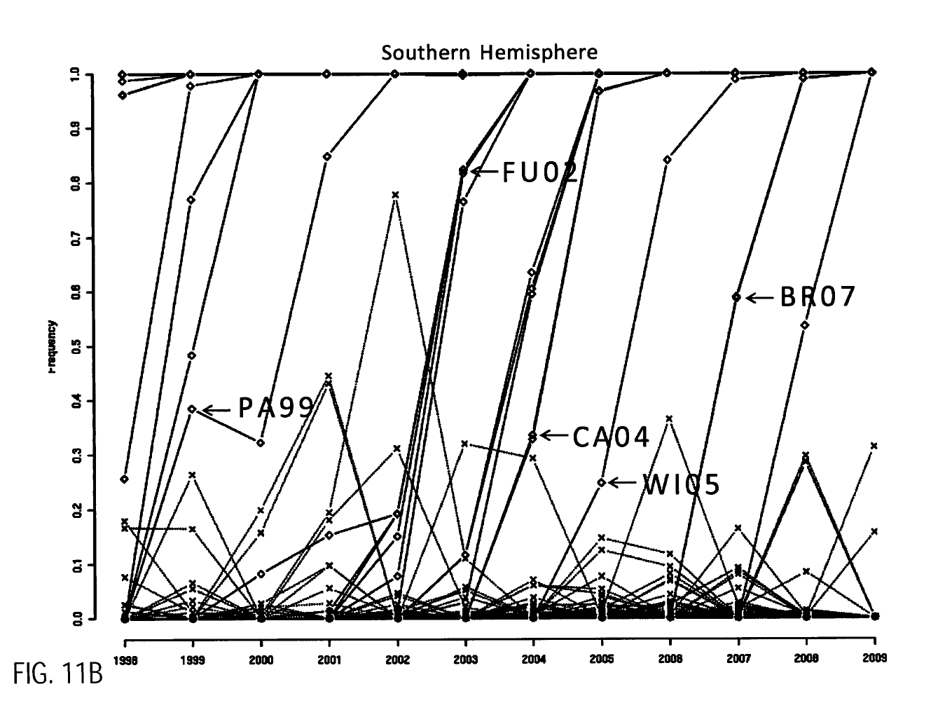

FIGS. 11A and 11B show gene allele predominance plots where alleles that reach a prevalence of more than 90% in frequency for a given year are shown in solid lines with data points given as open diamonds, whereas all others are shown in gray hatched lines with data points given as crosses. From these plots the allele which corresponds to the next predominant strain can be identified with good accuracy as the one with the steepest slope in comparison to the previous year.

Northern hemisphere allele selection (FIG. 11A): Predominant alleles are named according to literature names of predominant influenza A (H3N2) variants between 1998 and 2009. The PA99 alleles are first seen in 1998/99 and become predominant in 1999/2000. The FU02 are selected in 2001/02 or 2002/03, respectively, and became predominant in the season 2003/04. The first CA04 alleles were seen in 2003/04, but the final alleles are detected in 2004/05 when they are already predominant. WI05 alleles can be selected in 2005/06, one year before it becomes predominant. In 2006/07 BR07 alleles can be selected that become predominant in 2007/08. Thus, in 4 out of 5 cases, it is possible to select the strain that becomes predominant, one year before it becomes predominant, i.e. the predictive accuracy of the inventive method for the northern hemispheres is 80% (WHO prediction accuracy was 2 out of 6, see Table 6).

Southern hemisphere allele selection (FIG. 11B): The PA99 alleles are first seen in 1999, the same year when they became predominant. The FU02 are selected in 2002 and became predominant in the 2003 season. The first CA04 alleles were seen in 2004 and were predominant in 2005. WI05 alleles can be selected in 2005, one year before it becomes predominant in 2006. In 2007 BR07 alleles can be selected, but only as they were already predominant. Thus, the selection works in 3 out of 5 cases and the predictive accuracy of the inventive method for the southern hemisphere is around 60% (WHO prediction accuracy was 1 out of 5, see Table 6).

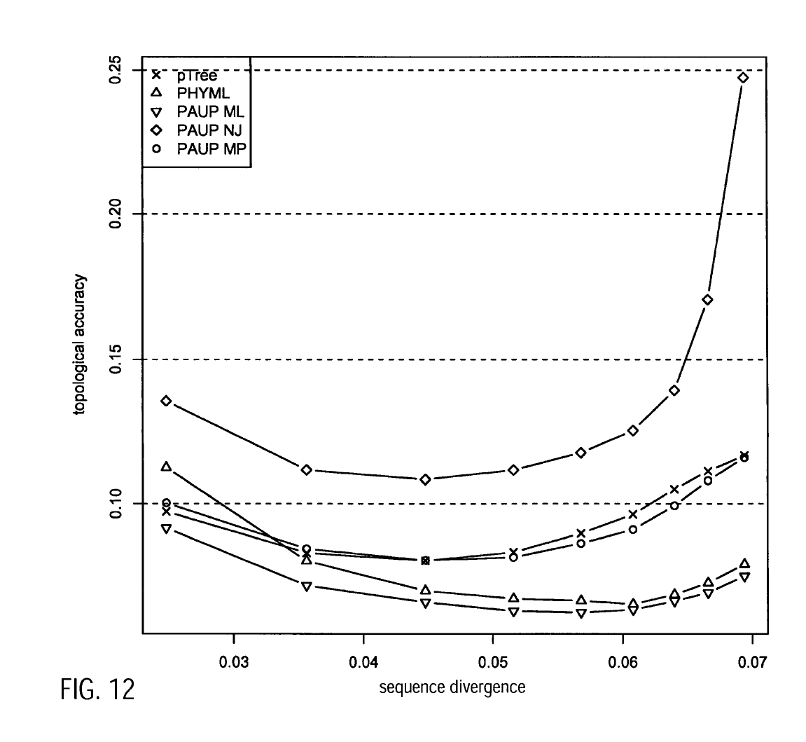

The predictive accuracy of the pTree method on benchmark data is also shown in FIG. 12.

The analysis of topological accuracy was done similar to (Guindon and Gascuel 2003, Systematic Biology 2: 696-704) on a benchmark data set of 5000 known tree topologies which are to be inferred from sequence data sets of 40 sequences each at a range of evolutionary distances. For comparison with pTree, we used the maximum likelihood method of PHYML (Guindon and Gascuel 2003) in combination with subsequent branch length optimization with Garli (D. Zwickl, 2006, “Genetic algorithm approaches for the phylogenetic analysis of large biological sequence datasets under the maximum likelihood criterion.” The University of Texas at Austin (PhD thesis)), the Neighbor Joining algorithm, the maximum likelihood method and the maximum parsimony method of PAUP (Swofford, D., 2002, PAUP* (2002). Phylogenetic Analysis Using Parsimony (*and other methods), Version 4, Sinauer Associates, Sunderland, Mass.). FIG. 12 shows a plot of the sequence divergence (maximum difference between two taxa in the data normalized by sequence length) versus the topological accuracy of the different methods as measured with the Robinson-Foulds (RF) distance (normalized by the number of internal edges of the trees) between the true and inferred tree topologies.

The results show that pTree has a similar accuracy as standard parsimony for sequence data sets across the range of evaluated divergence of the data, performs consistently better than the NJ algorithm, and is competitive to maximum likelihood approaches for data sets of a divergence of less than 0.35.

While the invention has been described with respect to the physical embodiments constructed in accordance therewith, it will be apparent to those skilled in the art that various modifications, variations and improvements of the present invention may be made in the light of the above teachings and within the purview of the appended claims without departing from the spirit and intended scope of the invention. In addition, those areas in which it is believed that those of ordinary skill in the art are familiar, have not been described herein in order to not unnecessarily obscure the invention described herein. Accordingly, it is to be understood that the invention is not to be limited by the specific illustrative embodiments, but only by the scope of the appended claims.

What is claimed is:

1. A method for identifying a phylogenetic tree for a plurality of biological sequences, each biological sequence being associated with a time stamp, the method comprising:
obtaining distances between biological sequences of the plurality of biological sequences;
generating a graph representation of the plurality of biological sequences based on the distances and the time stamps associated with the plurality of biological sequences;
identifying a minimum spanning tree (MST) of the generated graph as the phylogenetic tree.

2. The method of claim 1, wherein generating the graph representation comprises:
determining identical sequences in the plurality of biological sequences;
(ii) for each set of identical sequences, selecting a biological sequence from the set of identical sequences associated with the earliest time stamp;
(iii) selecting all unique biological sequences;
(iv) creating for each selected biological sequence of the plurality of biological sequences a node representing the selected biological sequence, each node being associated with the time stamp of the respective selected biological sequence; and
(v) adding an outgoing edge from a first node to a second node, if the time stamp associated with the second node is later than the time stamp associated with the first node.

3. The method of claim 2, wherein adding comprises:
determining the node representing the biological sequence associated with the earliest time stamp; and
adding outgoing edges from the determined node to all other nodes.

4. The method of claim 2, further comprising weighting each one of the added edges with a distance between the biological sequences represented by the nodes connected by the edge, the distance being derived from a distance matrix and wherein identifying the MST comprises:
selecting for each node the incoming edge with the lowest weight; and
removing all edges not selected.

5. The method of claim 2, further comprising:
(vi) identifying an initial MST from the generated graph;
(vii) inferring sequences of unsampled evolutionary intermediates based on differences between sequences represented by nodes connected by an edge in the initial MST;
(viii) assigning time stamps to the inferred sequences of unsampled evolutionary intermediates;
(ix) adding the inferred sequences to the plurality of biological sequences;
(x) repeating steps (i) to (ix) until no new unsampled evolutionary intermediates are inferred;
(xi) adding the sequences not selected at the iterations of step (ii) to the set of biological sequences being selected after step (x);
(xii) creating for each biological sequence of the set of biological sequences derived at step (xi) a node representing the biological sequence, each node being associated with the time stamp of the respective biological sequence; and
(xiii) adding an outgoing edge from a first node to a second node, if the time stamp associated with the second node is later than the time stamp associated with the first node;
wherein the MST identified as the phylogenetic tree is the MST identified in the graph generated at step (xiii).

6. The method of claim 2, further comprising:
if the time stamps associated with the plurality of biological sequences are specified with a resolution of a time span, connecting all nodes representing biological sequences associated with sampling dates within the same time span; and
resolving cycles in the generated graph during identification of the MST.

7. A system for identifying a phylogenetic tree for a plurality of biological sequences, each biological sequence being associated with a time stamp, wherein the system comprises:
(a) a data preparer adapted to obtain and output distances between biological sequences of the plurality of biological sequences; and
(b) a phylogeny identifier communicatively coupled to the data preparer, wherein the phylogeny identifier comprises: (i) a graph generator adapted to generate a graph representation of the plurality of biological sequences based on the output distances and the time stamps associated with the plurality of biological sequences; and (ii) a minimum spanning tree identifier adapted to compute a minimum spanning tree of the generated graph and identify the computed MST as the phylogenetic tree.

8. A method of preparing a vaccine against a fast-evolving entity; based on a phylogenetic tree, wherein the phylogenetic tree is a minimum spanning tree of a graph representation of a plurality of biological sequences generated based on distances between the biological sequences and time stamps associated with the plurality of biological sequences, wherein the method comprises:
selecting an individual isolate of the fast-evolving entity; and
(ii) preparing a vaccine based on the individual isolate selected in (i).

9. The method of claim 8, wherein the selecting of an individual isolate of the fast-evolving entity comprises the steps of:
(a) identifying the number of repeated amino acid mutations occurring in the isolate sequence; and/or
(b) identifying the position of the isolate in the phylogenetic tree; and
(c) selecting an isolate as a vaccine candidate, wherein the isolate sequence bears the maximum number of repeated amino acid mutations among all tested sequences which cluster in the phylogeny, occurred within a time span of 4 years before the season in which the vaccine is to be produced, and are located within the antigenic sites of the protein.

10. The method of claim 8, wherein the sequences are selected from the group consisting of RNA, DNA, and protein sequences.

11. The method of claim 8, wherein the fast-evolving entity is a virus.

12. The method of claim 11, wherein the virus is an influenza virus.

13. The method of claim 8, wherein the phylogenetic tree is established using nucleic acid sequences encoding, or protein sequences of, hemagglutinin (HA) or neuraminidase (NA).

14. A vaccine produced by a method of preparing a vaccine against a fast-evolving entity based on a phylogenetic tree, wherein the phylogenetic tree is a minimum spanning tree of a graph representation of a plurality of biological sequences generated based on distances between the biological sequences and time stamps associated with the plurality of biological sequences, wherein the method comprising the steps of (i) selecting an individual isolate of the fast-evolving entity and (ii) preparing a vaccine based on the individual isolate selected in (i).

15. A method selecting an influenza vaccine candidate, comprising the steps of:
(a) inferring a phylogenetic tree by obtaining distances between biological sequences of a plurality of biological sequences from influenza isolates,
(b) generating a graph representation of the plurality of biological sequences based on the distances and time stamps associated with the plurality of biological sequences and identifying a minimum spanning tree (MST) of the generated graph as the phylogenetic tree,
(c) inferring a gene predominance plot showing the frequencies of gene alleles in the analyzed viral population over time, and
(d) identifying the vaccine candidate as the isolate having the steepest slope in the gene predominance plot in comparison to the previous year.

16. The method according to claim 15, wherein the frequency of gene alleles in one year is the relative number of isolates in a subtree of the branch based on which the gene allele is defined.

17. A method of selecting an influenza vaccine candidate, which method comprises:
(a) identifying a phylogenetic tree by obtaining distances between biological sequences of a plurality of biological sequences from influenza isolates,
(b) generating a graph representation of the plurality of biological sequences based on the distances and time stamps associated with the plurality of biological sequences and identifying a minimum spanning tree (MST) of the generated graph as the phylogenetic tree,
(c) identifying the number of repeated amino acid mutations occurring in each isolate sequence, and
(d) selecting an isolate as a vaccine candidate, wherein
(i) the isolate sequence bears the maximum number of repeated amino acid mutations that occurred within a time-span of 4 years before the season in which the vaccine is to be produced,
(ii) the isolate sequence bears the maximum number of repeated amino acid mutations that are located in antigenic sites and occurred within a time-span of 4 years before the season in which the vaccine is to be produced,
(iii) the isolate sequence bears the maximum number of repeated amino acid mutations that cluster in the phylogeny, are located in antigenic sites and occurred within a time-span of 3 years before the season in which the vaccine is to be produced, or
(iv) the isolate sequence bears the maximum number of repeated amino acid mutations that cluster in the phylogeny, are located in sites determined experimentally to change the viral phenotype and occurred within a time-span of 4 years before the season in which the vaccine is to be produced.
